(19) United States
(12) Patent Application Publication
(10) Pub. No.: US 2008/0187908 A1
(43) Pub. Date: Aug. 7, 2008

(54) Granulocyte Subtype-Selective Receptors And Ion Channels And Uses Thereof

(76) Inventor: Chaker N. Adra, Boston, MA (US)

Correspondence Address: WOLF GREENFIELD & SACKS, P.C., 600 ATLANTIC AVENUE, BOSTON, MA, 02210-2206, US

(21) Appl. No.: 10/591,628

(22) PCT Filed: Mar. 3, 2005
(86) PCT No.: PCT/US2005/007519

§ 371 Date: Nov. 16, 2007

Related U.S. Application Data
(60) Provisional application No. 60/549,865, filed on Mar. 3, 2004.

(51) Int. Cl. C12Q 1/68; G01N 33/53

(52) U.S. Cl. 435/6; 435/7.1

(57) ABSTRACT

The invention relates, in part, to granulocyte type selective markers that are preferentially expressed in granulocytes and their use in drug screening assays. Additionally, the granulocyte type selective markers are useful in the diagnosis and treatment of granulocyte disorders and in the assessment of the efficacy of therapies of granulocyte disorders.

### FIELD OF THE INVENTION

The present invention relates to the identification of granulocyte-selective markers that can be used as targets for drug discovery.

### BACKGROUND OF THE INVENTION

Three types of human blood granulocytes, eosinophils, basophils and neutrophils, play roles in protecting against microbial infection by releasing cell type-specific mediators and proteases. Specifically, eosinophils and basophils evoke allergic reactions as well as damage nematodes.^{1,2} As well as killing bacteria, neutrophils sometimes induce systemic vasculitis or multiple organ damage under certain conditions.^{3,4} Thus, targeting granulocyte type-selective functions is considered an important strategy for drug discovery.

Activation of blood granulocytes and tissue mast cells is generally characterized by an influx of extracellular calcium (Ca²⁺), which is essential for subsequent release of granule-derived mediators, newly generated lipid mediators and cytokines.⁵ The mechanism by which granulocyte mediator secretion is sustained is therefore likely to include modulation of various types of ion channels. Flow of ions including K⁺ and Cl⁻ may play an important role during granulocyte responses because they regulate cell membrane potential and thus influence Ca²⁺ influx.⁶ Treatment of mast cells and basophils with pertussis toxin inactivates the Gi-type of G-proteins and abolishes degranulation induced by non-immunological ligands such as thrombin and N-formylpeptide; however, it fails to inhibit the influx of Ca²⁺.⁷ Thus, Ca²⁺-independent stimulation of Gi is also involved in granulocyte degranulation. The thrombin (protease) activated receptors and formylpeptide receptors are classified as G protein-coupled receptors (GPR), having an extracellular N-terminal segment, a seven transmembrane region, which forms the transmembrane core; three exoloops, three cytoloops, and a C-terminal segment.⁸ Thus, ion channels and GPR both play essential roles in degranulation as well as other cellular function important for granulocytes. As a result, both ion channels and GPR are targets of drug development.⁹

As the human genome project nears completion, the identification of potential drug targets using gene expression profiles from specific cell types is becoming practical and important for drug discovery.^{10,11} The sequencing of the human genome is offering an unprecedented opportunity for the pharmaceutical development of drugs. Receptor genes and ion channel genes are found only in 5% and 1.3% of all genes present in the human genome,¹⁰ respectively. However, receptors and ion channels are respectively found in 45% and 5% of the molecular targets of all known drugs.^{9,12,13} Thus, receptors including GPR and ion channels are now considered as the most important drug targets.

Until recently, it has been impractical to analyze genome-wide expression of leukocytes. Newly developed technology, the microarray or high density oligonucleotide probe array (GeneChip) is one of the latest breakthroughs in experimental molecular biology, which allows approximately 39,000 transcripts derived from a cells transcriptome to be simultaneously monitored. Using this technology, we previously reported the transcriptome profiling of various types of mast cells and eosinophils.¹⁴⁻¹⁶ However, there is still a need in the art to identify drug targets that are selectively, or preferentially, expressed in specific cell types such that efforts required for pharmaceutical development are minimized.

### SUMMARY OF THE INVENTION

In the present study, we used GeneChip (version U133A containing approximately 22,000 gene probes) to examine the granulocyte type-selective transcriptome expression of 7 types of leukocytes (basophils, eosinophils, neutrophils, CD4⁺ cells, CD8⁺ cells, CD14⁺ cells and CD19⁺ cells), platelets, mast cells and fibroblasts by focusing on the expression of granulocyte-selective genes for ion channels, GPR and other receptors. We identified many novel granulocyte subtype-selective transcripts (markers) that are useful for drug development.

Granulocyte subtype selective transcripts were chosen based on several conditions such as the transcript having 5-fold or greater expression level compared to the maximum level of other leukocytes. Fifty-one transcripts were chosen to be preferentially expressed by each granulocyte subtype. Seventeen out of the 51 transcripts have not been previously reported as granulocyte subtype-selective. Among the 17 receptors and ion channels, six were basophil- and/or eosinophil-selective and were not highly expressed by other organs, indicating that they may be potential targets for anti-allergic drugs, for example.

Utilization of this database of potential granulocyte type-selective drug targets will minimize the efforts required for pharmaceutical development of drugs for treatment of diseases of the immune system, cancer, cardiac diseases, as well as other diseases.

Accordingly, the invention provides methods and compositions that are useful for drug discovery, disease diagnosis and/or prognosis, granulocyte type detection and/or selection and/or manipulation, and/or therapeutic applications.

Accordingly, in one aspect, methods of the invention include diagnosing a granulocyte disorder by detecting, in a biological sample obtained from a subject, a level of expression of one or more granulocyte-selective markers, and comparing the level of expression of each of the one or more granulocyte-selective markers with a reference level of expression. A statistically significant difference between the level of expression of at least one granulocyte-selective marker and an expected level of expression for the at least one granulocyte-selective marker is indicative of a granulocyte disorder in the subject. The reference level of expression for a granulocyte-selective marker may be, for example, a normal level of expression of the granulocyte-selective marker in a normal granulocyte. A higher level of expression of at least one of the one or more granulocyte-selective markers in the biological sample compared to the expected level of expression for the at least one granulocyte-selective marker may be indicative of the granulocyte disorder. A lower level of expression of at least one of the one or more granulocyte-selective markers in the biological sample compared to the expected level of expression for the at least one granulocyte-selective marker also may be indicative of the granulocyte disorder. In one embodiment, the granulocyte disorder may be an abnormally high number of one or more types of granulocyte in the biological sample. In another embodiment, the granulocyte disorder may be an abnormally low number of one or more types of granulocyte in the biological sample. In yet another embodiment, the granulocyte may be an abnormal pattern of expression of one or more granulocyte selective markers in one or more types of granulocyte in the biological sample.

In another aspect, methods of the invention include diagnosing a non-neutrophil granulocyte disorder or mast cell disorder by detecting, in a biological sample from a subject, a level of expression of one or more non-neutrophil granulocyte or mast cell selective markers, and comparing the level of expression of each of the one or more non-neutrophil granulocyte or mast cell selective markers with a reference level of expression. A statistically significant difference between the level of expression of at least one non-neutrophil granulocyte or mast cell selective marker and an expected level of expression for the at least one non-neutrophil granulocyte or mast cell selective marker may be indicative of a non-neutrophil granulocyte disorder or mast cell disorder in the subject. In one embodiment, the non-neutrophil granulocyte disorder may be a basophil disorder. The basophil disorder may be one or more of the diseases described herein, including a tumor or cancer. In another embodiment, the non-neutrophil granulocyte disorder may be an eosinophil disorder. The eosinophil disorder may be one or more of the diseases described herein, including a tumor or cancer. In other embodiments, a mast cell disorder may be one or more of the diseases described herein, including a tumor or cancer.

A higher level of expression of one or more non-neutrophil granulocyte or mast cell-selective marker in the biological sample compared with the control level of expression of the one or more non-neutrophil granulocyte or mast cell-selective marker may be diagnostic of the non-neutrophil granulocyte disorder or mast cell disorder. Alternatively, a lower level of expression of one or more non-neutrophil granulocyte or mast cell-selective marker in the biological sample compared with the control level of expression of the one or more non-neutrophil granulocyte or mast cell-selective marker also may be diagnostic of the non-neutrophil granulocyte disorder or mast cell disorder.

In another aspect, methods of the invention include determining onset, progression, or regression, of a granulocyte disorder in a subject, by i) detecting in a first biological sample from a subject a first level of expression of one or more granulocyte-selective markers, ii) detecting in a second biological sample comprising blood and obtained from the subject at a time later than the first biological sample, a second level of expression of the one or more granulocyte-selective markers, and iii) comparing the first level of expression with the second level of expression. A statistically significant difference between the first and second levels may be an indication of onset, progression, or regression of the granulocyte disorder.

In another aspect, methods of the invention include selecting a course of treatment for a subject having or suspected of having a granulocyte disorder by i) detecting in a biological sample from a subject a level of expression of one or more granulocyte-selective markers, ii) comparing the level of expression of the one or more granulocyte-selective markers to a reference level of expression, iii) determining the status of the granulocyte disorder of the subject based on the difference in the level of expression of one or more granulocyte-selective marker in the sample compared to the reference level of expression, and iv) selecting a course of treatment for the subject appropriate to the status of the granulocyte disorder of the subject.

In another aspect, methods of the invention includes monitoring responses to treatment in a subject with a granulocyte disorder by i) detecting in a biological sample from a subject that has received treatment for the granulocyte disorder, a level of expression of one or more granulocyte-selective markers, and ii) comparing the level of expression of the one or more granulocyte-selective marker with a reference level of expression. A statistically significant change in the level of expression of one or more of the granulocyte-selective markers in the biological sample relative to the reference level of expression may indicate that the subject is responding to the treatment for the granulocyte disorder.

In another aspect, methods of the invention include identifying a compound that alters at least one physiological property of a granulocyte by i) contacting a granulocyte with a candidate compound that interacts with a granulocyte-selective marker, ii) determining at least one physiological property of the granulocyte after contact with the candidate compound, and iii) comparing the at least one physiological property to one at least one reference property to determine whether the candidate compound alters at least one physiological property of the granulocyte. In one embodiment, the effect of the candidate compound on a granulocyte may be compared to its effect on a second cell type to determine whether the candidate compound has a cell-type selective effect on the granulocyte. The second cell type may be another granulocyte cell type. Alternatively, the second cell type may be a non-granulocyte cell type (including a mast cell or other leukocyte). In one embodiment, a physiological property may be an expression level of one or more granulocyte-selective markers. In another embodiment, a physiological property may be the growth rate and/or proliferation rate of the granulocyte. In one embodiment, the candidate compound may be cytostatic or cytotoxic (e.g., it may kill the granulocyte). It should be appreciated that the candidate compound may have cell type selective effects (e.g., cell type selective cytotoxic and/or cytostatic effects).

In another aspect, methods of the invention include treating a granulocyte-associated disease, by administering to a subject having a granulocyte-associated disease a compound that interacts with a granulocyte-selective marker in an amount sufficient to treat the granulocyte-associated disease. Similarly, the invention provides methods for treating a mast cell-associated disease by administering to a subject having a mast cell-associated disease a compound that interacts with a mast cell-selective marker in an amount sufficient to treat the mast cell-associated disease. In some embodiments, a plurality (e.g., any whole number between 1 and 50, for example 1, 2, 3, 4, 5, 6, 7, 8, 9, 10, or more) of granulocyte-selective or mast cell-selective compounds may be administered. Compounds may be administered in physiologically acceptable (e.g., biologically compatible) solutions or carrier. Therapeutic preparations of the invention may be sterilized using any appropriate technique including filtration, heat treatment, radiation, chemical treatment, and/or other suitable techniques.

In another aspect, methods of the invention include treating a granulocyte-associated disease by modulating the activity or expression of a granulocyte-selective marker to an extent that is sufficient to treat the granulocyte-associated disease. Similarly, the invention also provides methods of treating a mast cell-associated disease by modulating the activity or expression of a mast cell-selective marker thereby to treat the mast cell-associated disease. Modulating the activity or expression of a marker may involve activating or inactivating and/or increasing or decreasing the expression levels.

In any of the therapeutic methods described herein, one or more therapeutic compounds may be identified as described herein by screening for one or more compounds that alter at least one physiological property of a granulocyte and/or a mast cell.

In another aspect, the invention provides compounds that alter a physiological property of a granulocyte and/or a mast cell. One or more compounds may be identified as described herein by screening compounds that are known to interact (e.g., specifically, selectively, etc.) with a granulocyte-selective marker and/or a mast cell-selective marker. Compounds of the invention may be formulated in a physiologically acceptable preparation.

In any of the aspects described herein, the level of expression of each of the one or more granulocyte-selective markers (including neutrophil-selective markers, basophil-selective markers, eosinophil-selective markers, or a combination thereof) may be determined by determining an amount of an mRNA in the biological sample corresponding to each of the one or more granulocyte-selective markers. The amount of mRNA may be determined by reverse transcription polymerase chain reaction (RT-PCR) amplification or any other suitable method including microarray analysis or other hybridization or primer extension assays (e.g., using labeled probes and/or nucleotides). In other embodiments, the level of expression of each of the one or more granulocyte-selective markers may be determined by determining an amount of a protein in the biological sample corresponding to each of the one or more granulocyte-selective markers (e.g., using antibody binding assays with labeled antibodies, including ELISA assays or other antibody binding assays). Other suitable methods of determining expression levels of granulocyte-selective markers may be used (including activity levels). Similarly, expression levels of non-neutrophil granulocyte or mast cell-selective markers may be determined by determining mRNA and/or protein levels or using any other appropriate assay (including functional assays).

In any of the aspects described herein, the biological sample may be a blood sample, a tissue sample, or any other suitable biological sample.

In any of the aspects described herein, the subject may be human.

In any of the aspects described herein, a granulocyte may be a neutrophil, a basophil, or an eosinophil. It should be appreciated that aspects of the invention described herein in the context of granulocytes may be applied to non-neutrophil granulocytes and/or mast cells. Similarly, aspects of the invention described herein in the context of granulocytes or mast cells may be applied to other leukocytes including those described in the examples and for which cell-type selective expression is shown in the experiments including the Figures attached hereto.

According to aspects of the invention, one or more methods described herein may involve a combination of two or more markers. In one embodiment, markers that are more selective as described herein may be used. In some embodiments, a combination of two or more (e.g., 3, 4, 5, 6, 7, 8, 9, or 10 or more) markers may be used to increase the cell-selectivity of a screening or diagnostic method. It should be appreciated that different combinations of markers disclosed herein may be used (e.g., panels of two or more markers). Accordingly, compositions of the invention may include compounds that interact with two or more markers (e.g., a single compound that interacts with two or more markers, or two or more compounds each of which interacts with a single marker, or any combination thereof).

Methods and compositions described herein may include or involve nucleic acids that hybridize to one or more nucleic acids that encode a granulocyte or mast cell-selective marker (e.g., DNA or RNA). The nucleic acids may be oligonucleotides (e.g., synthetic oligodeoxynucleotides). The oligonucleotides may be between 10 and 100 nucleotides in length (e.g., between about 20 and about 50). The oligonucleotides may include sequences that are complementary to sequences of one or more markers described herein (or the complement thereof). In some embodiments, nucleic acids may be antisense or siRNA nucleic acids that are useful to decrease the expression of one or more granulocyte or mast cell-selective markers. Similarly, methods and compositions described herein may include or involve agents that bind or interact with one or more proteins that are granulocyte or mast cell-selective markers. Binding agents may be antibodies or fragments thereof (including single chain antibodies, synthetic antibodies, humanized antibodies), aptamers, and/or other molecules including naturally occurring or synthetic molecules (e.g., low molecular weight molecules), that bind to one or more peptide epitopes of one or more granulocyte and/or mast cell-selective marker. Examples of nucleic acid and peptide sequences for certain granulocyte and/or mast cell-selective markers are provided in the sequence listing. These and others are described in the examples, tables, and figures. In the sequence listing, the nucleic acid sequences are presented as cDNA sequences. The following list of sequences in the sequence listing includes the description of the molecule and the genbank accession number is indicated in parentheses.

| | |
|---|---|
| SEQ ID NO: 1 | Ca²⁺ channel type A1 D (BE550599) cDNA |
| SEQ ID NO: 2 | K⁺ channel Kir1.3 (U73191.1) cDNA |
| SEQ ID NO: 3 | K⁺ channel Kir1.3 (U73191.1) translation |
| SEQ ID NO: 4 | K⁺ channel Kir2.1 (AF153820.1) cDNA |
| SEQ ID NO: 5 | K⁺ channel Kir2.1 (AF153820.1) translation |
| SEQ ID NO: 6 | PGE R type 3a2 (X83858.1) cDNA |
| SEQ ID NO: 7 | PGE R type 3a2 (X83858.1) translation |
| SEQ ID NO: 8 | EMR-1 (NM_001974.1) cDNA |
| SEQ ID NO: 9 | EMR-1 (NM_001974.1) translation |
| SEQ ID NO: 10 | GPR105 purinergic R (NM_014879.1) cDNA |
| SEQ ID NO: 11 | GPR105 purinergic R (NM_014879.1) translation |
| SEQ ID NO: 12 | GPR, Edg-4 (AF011466.1) cDNA |
| SEQ ID NO: 13 | GPR, Edg-4 (AF011466.1) translation |
| SEQ ID NO: 14 | PAR1-like GPR43 (NM_005306.1) cDNA |
| SEQ ID NO: 15 | PAR1-like GPR43 (NM_005306.1) translation |
| SEQ ID NO: 16 | GPR77 (018485.1) cDNA |
| SEQ ID NO: 17 | GPR77 (018485.1) translation |
| SEQ ID NO: 18 | GPR86 purinergic R (NM_023914.1) cDNA |
| SEQ ID NO: 19 | GPR86 purinergic R (NM_023914.1) translation |
| SEQ ID NO: 20 | PAR2 (BE965369) cDNA |
| SEQ ID NO: 21 | HTm4 (L35848.1) cDNA |
| SEQ ID NO: 22 | HTm4 (L35848.1) translation |
| SEQ ID NO: 23 | CD244 NK cell R (NM_016382.1) cDNA |
| SEQ ID NO: 24 | CD244 NK cell R (NM_016382.1) translation |
| SEQ ID NO: 25 | Fibroblast growth factor R 2 (NM_022969.1) cDNA |
| SEQ ID NO: 26 | Fibroblast growth factor R 2 (NM_022969.1) |
| | translation |
| SEQ ID NO: 27 | Low-density lipoprotein R (NM_000527.2) cDNA |
| SEQ ID NO: 28 | Low-density lipoprotein R (NM_000527.2) translation |
| SEQ ID NO: 29 | Butyrophilin-like R (AK025267.1) cDNA |
| SEQ ID NO: 30 | Leukocyte Ig-like R A2 (NM_006866.1) cDNA |
| SEQ ID NO: 31 | Leukocyte Ig-like R A2 (NM_006866.1) translation |

### BRIEF DESCRIPTION OF THE DRAWINGS

FIG. 1 shows Real-time quantitative PCR analysis of granulocyte-selective gene expression. The relative mRNA expression level by each cell type against PBMNC was shown after normalization of mRNA levels for A. HTm4 (0.42 per 100 GAPDH), B. Ca²⁺ receptor alpha 1D subunit (0.003), C. prostaglandin E receptor type 3a2 (0.12), D. EMR-1 (0.62), and E. aquaporin 9 (0.92) expressed by PBMNC. Ne; neutrophils (n=3), Eo; eosinophils (n=2), Ba, basophils (n=3), CD4; CD4+ cells (n=3), P; PBMNC (n=1).

FIG. 2 shows the demonstration of HTm4 protein on human basophils. Cells on the glass slide were incubated with 2 μg/ml polyclonal rabbit anti-hHTm4 antibody or 2 μg/ml rabbit IgG (H+L) as a control followed by incubation with a secondary antibody, highly cross-adsorbed Alexa Fluor® 546 conjugated goat anti-rabbit IgG (H+L). After mounting using the Prolong AntiFade Kit, slides were scanned by Zeiss Laser Scanning Microscope 5 Pascal.

FIGS. 3A through 3E shows granulocyte subtype-specific transcripts for ion channels and receptors.

FIGS. 4A through 4F show a table of “normalized AD” expression levels of various genes in indicated cells and shows corresponding graphs; x axis represents “normalized AD” expression levels.

FIGS. 5A through 5D show a table of “normalized AD” levels of various genes in indicated cells and shows corresponding graphs; x axis represents “normalized AD” expression levels.

FIG. 6A through FIG. 6R show the complete list of granulocyte subtype-selective transcripts.

### DETAILED DESCRIPTION OF THE INVENTION

In one aspect of the invention, one or more granulocyte-selective markers may be used for drug discovery. For example, a granulocyte-selective marker (e.g., a cell type selective transcript, its protein product, or the gene encoding the marker) may be used as a target for drug identification. Drug identification may involve a random screen of potential drug candidates (including peptides, nucleic acids, small molecules, etc.). In one embodiment, one or more granulocyte-selective markers may be used as targets for drug identification. For example, a single drug molecule that interacts (e.g., that activates, or inactivates, and/or binds to) with one or more markers may be identified. However, in some embodiments, more than one drug molecule may be identified, each one interacting with a different granulocyte-selective marker. According to aspects of the invention, drug(s) that target a specific granulocyte may be used to stimulate or inhibit activity or growth of that cell type. A cell type specific drug also may be used to kill or slow or stop the growth of a specific cell type.

Examples of markers for different granulocyte types include: Ca²⁺ channel type A1 D (GenBank accession no. BE550599), K⁺ channel Kir1.3 (GenBank accession no. U73191.1), K⁺ channel Kir2.1 (GenBank accession no. AF153820.1), PGE R type 3a2 (GenBank accession no. X83858.1), EMR-1 (GenBank accession no. NM_{—}001974.1), GPR105 purinergic R (GenBank accession no. NM_{—}014879.1), GPR, Edg-4 (GenBank accession no. AF011466.1), PAR1-like GPR43 (GenBank accession no. NM_{—}005306.1), GPR77 (GenBank accession no. NM_{—}018485.1), GPR86 purinergic R (GenBank accession no. NM_{—}023914.1), PAR2 (GenBank accession no. BE965369), HTm4 (GenBank accession no. L35848.1), CD244 NK cell R (GenBank accession no. NM_{—}016382.1), Fibroblast growth factor R 2 (GenBank accession no. NM_{—}022969.1), Low-density lipoprotein R (GenBank accession no. NM_{—}000527.2), Butyrophilin-like R (GenBank accession no. AK025267.1), and Leukocyte Ig-like R A2 (GenBank accession no. NM_{—}006866.1).

Some of these markers are specific for one cell type only (e.g., HTm4 (GenBank accession no. L35848.1) is preferentially expressed in basophils, and GPR105 purinergic R (GenBank accession no. NM_{—}014879.1) is preferentially expressed in eosinophils). Others are preferentially expressed in two or three cell types (e.g., Ca²⁺ channel type A1 D (GenBank accession no. BE550599), and EMR-1 (GenBank accession no. NM_{—}001974.1) are expressed in both basophils and eosinophils.

In one aspect, the invention provides methods for screening compounds to identify those that interact with one or more cell type selective markers disclosed herein. Cell type selective markers of the invention include receptors, ion channels (including calcium channels) and other molecules. Accordingly, candidate drug compounds may include one or more receptor binding compounds, ion channel binding compounds (including calcium channel binding compounds) and compounds that bind to one or more of the other molecules. Candidate drug compounds also may include compounds that can be transported across/through one or more ion channels. Candidate drug compounds also may include compounds that block ion transport (e.g., calcium transport).

In one embodiment, drug compounds may be agents that selectively or specifically bind to one or more cell type selective markers of the invention. For example, drug compounds may be antibodies or aptamers that bind to one or more cell type selective markers of the invention. A marker binding drug may directly inactivate the marker. Alternatively, a marker binding agent may activate the marker. In another embodiment, a marker binding agent may be used to target a second compound to cells that selectively express that marker. For example, the marker binding agent may be conjugated to one or more additional moieties to alter the physiology of the target cell (e.g., to activate, inactivate, or kill the target cell). For example, radioactive moieties, heavy metal moieties, certain enzymes, toxins, and other toxic compounds (e.g., other cytostatic or cytotoxic compounds) may be used to inactivate or kill a target cell. In contrast, activating moieties (e.g., cytokines, growth factors, etc.) may be used to activate a target cell. In certain embodiments, a binding agent (e.g., an antibody, aptamer, etc.) may be bispecific, trispecific, or multispecific (meaning that it binds to two, three, or more cell selective markers). Multispecific binding agents may be better targeting moieties if they bind to a specific combination of markers that is selectively or specifically present on the target cell type. Preferred markers for binding agents are those that are accessible to the binding agents (e.g., extracellular, membrane bound, or transmembrane proteins). However, any of the markers described herein (or combinations thereof) may be used as the invention is not limited in this respect. Antibodies to one or more leukocyte (e.g., granulocyte, granulocyte subtype, mast cell, etc.) marker may be used in several aspects of the invention. Antibodies may be monoclonal, polyclonal, humanized, synthetic, single-chained, etc., or any useful combination thereof. Selective marker binding agents also may be conjugated to one or more detection moieties (e.g., fluorescent, radioactive, enzyme mediated, etc.) and used as detection agents to detect one or more leukocyte cell types.

In another aspect of the invention, one or more granulocyte-selective markers may be used for diagnostic and/or prognostic applications. For example, a single cell type-selective marker may be used as a marker of the presence of a specific granulocyte type-selective marker in diseased tissue. Similarly, a panel of granulocyte-selective markers (e.g., 1, 2, 3, 4, 5, 6, 7, 8, 9, 10, etc.) may be used as markers indicating the presence of a specific granulocyte-selective marker in diseased tissue (e.g., in a tumor or cancerous growth). Accordingly, any one or more of Ca²⁺ channel type A1 D (GenBank accession no. BE550599), EMR-1 (GenBank accession no. NM_{—}001974.1), or GPR105 purinergic R (GenBank accession no. NM_{—}014879.1) may be used to detect, for example, the presence of eosinophils in a biological sample. Similarly, one or more markers of the invention may be used to determine the ratio of different granulocyte types in a biological tissue or sample from a subject.

A biological sample includes, but is not limited to: cells, tissue, body fluid (e.g. blood, serum, etc.). The tissue may be in a subject, obtained from a subject, or may be grown in culture (e.g. from a cell line). A cell or tissue used in the invention can be a blood cell or other cell type. As used herein, cell samples, tissue samples, and/or blood samples, etc., may be obtained using methods well known to those of ordinary skill in the related medical arts. A biological sample may be, for example, a tissue biopsy sample or a biological fluid or solid.

As used herein, a subject is a mammal, preferably a human, non-human primate, cow, horse, pig, sheep, goat, dog, cat, or rodent. In all embodiments, human subjects are particularly preferred. In some embodiments, the subject is suspected of having a granulocyte disorder. In other embodiments the subject has been diagnosed with a granulocyte disorder.

The detection of one or more particular granulocyte types or granulocyte-selective markers in a biological sample, or the determination of an abnormal ratio of different cell types in a biological sample, may be useful for disease diagnostic or prognostic purposes. Allergies, cancers and infections are some examples of diseases characterized by an altered proliferation of one or more granulocyte cell type(s). Granulocyte disorders include basophil cell disorders and eosinophil cell disorders. Examples of basophil cell disorders include myeloproliferative disorders such as polycythemia vera and myelofibrosis.

Examples of basophil cell disorders include asthma, eosinophilic pneumonia, helminthic infestations and Eosinophilic Gastrointestinal Disorders. Examples of Eosinophilic Gastrointestinal Disorders include Eosinophilic Esophagitis (EE), Eosinophilic Gastritis (EG), Eosinophilic Duodenitis (ED), Eosinophilic Colitis (EC), Eosinophilic Gastroenteritis (EGE), and Eosinophilic Ileitis (El).

Diagnosis and treatment of mast cell disorders are also encompassed by this invention. Examples of Mast Cell Disorders include Systemic Mastocytosis (with or without cutaneous manifestations such as Urticaria Pigmentosa) such as Aggressive Mastocytosis, Indolent Mastocytosis, Mastocytosis with associated Hematologic Disorder, Mast Cell Leukemia, Cutaneous Mastocytosis such as Urticaria Pigmentosa (UP), Telengiecstasia Macularis Eruptive Perstans (TMEP), Mast Cell Activation Syndrome/Disorder, and Pediatric mast cell disorders such as Solitary Mastocytoma, Urticaria Pigmentosa, and Diffuse Cutaneous Mastocytosis.

In some embodiments, the detection of an abnormal level of one of more granulocyte-selective marker(s) may be used to determine appropriate drug or therapeutic regimens (e.g., abnormal growth of a particular cell type may suggest the use of one or more drugs that inhibit that growth of, or kill, that particular cell type).

In another aspect, diagnostic methods of the invention may be used to determine whether an abnormal expression pattern of one or more granulocyte-selective marker(s) is present on a particular granulocyte type. An abnormal pattern may be indicative of a disease (e.g., cancer, allergic response, infection, etc.). An abnormal pattern of expression of a granulocyte-selective marker may be an increase or decrease in the level of expression (at the level of transcription, translation, activity, or a combination of two or more thereof) of one or more granulocyte-specific markers relative to a reference level of expression.

The reference level may be a level of the same marker(s) in a reference cell or tissue. The reference level may be a level of one or more other granulocyte-selective marker(s). The reference level may the level of expression of one or more markers that are not granulocyte-selective. The reference level may be a predetermined control value. The reference level may be a combination of two or more of the above.

Levels of expression may be compared to controls. The control may be a predetermined value, which can take a variety of forms. It can be a single value, such as a median or mean. A control value can be established based upon comparative groups (e.g. comparative cell types), such as in cells having normal levels of expression of one or more granulocyte-selective marker. These types of control values can serve as control values for substantially similar cells that are contacted with a treatment compound. In some embodiments of the invention, a control level of expression of a granulocyte selective marker(s) is the level of expression of the granulocyte selective marker(s) in a non-granulocyte cell or tissue.

In a further aspect of the invention, one or more granulocyte-selective markers may be used for specific cell type detection, selection, and or manipulation. For example, an antibody or specific binding agent (e.g., aptamer etc.) that binds to a granulocyte-selective marker (e.g., a protein, including a membrane bound or a surface protein) may be detectably labeled (e.g., with a fluorescent label, a radioactive label, or any other detectable label or combination thereof) and used to bind to a particular cell type. The detectably labeled antibody (or other binding agent) may be used to detect a particular cell type directly in vivo (e.g., via an MRI, PET, or CAT scan or other in vivo imaging technique) or ex vivo (e.g., via microscopy such as fluorescent microscopy or other ex vivo imaging technique). The detectably labeled antibody (or other binding agent) may be used to detect the presence of markers indicative of particular cell types (e.g., in an assay for nucleic acid, protein, and/or activity levels performed on a processed sample obtained from a biological sample that includes cells or cellular debris). The detectably labeled antibody (or other binding agent) may be used to select (for or against) the presence of certain granulocyte cell types in a cell preparation (e.g., via a cell sorting technique such as FACS). Accordingly, granulocyte or cell preparations may be enriched or depleted for one or more granulocyte subtypes (e.g., basophil, neutrophil, or eosinophil) or mast cells using methods of the invention. In one embodiment, stem cell preparations may be enriched or depleted for one or more types of granulocyte or granulocyte precursor using one or more granulocyte-selective markers described herein.

In another aspect, markers of the invention may be used as targets for molecules that alter the function and development of certain granulocyte subtypes (e.g., may change their development path and direct them from one type of granulocyte to another, particularly for stem cells and other precursor cells).

In yet another aspect of the invention, one or more granulocyte-selective markers may be useful for therapeutic applications and particularly for cell type selective or specific therapies. This can be done by targeting one or more of the granulocyte-selective markers. Accordingly, one or more compounds that bind or interact with one or more cell type-specific markers may be used therapeutically. Treatment methods of the invention may be therapeutic (e.g., they reduce or cure symptoms of a disease) or prophylactic (e.g., they prevent symptoms of the disease).

The invention also provides for methods for selecting a course of treatment of a subject having or suspected of having a granulocyte disorder. In this aspect of the invention, the selection of the course of treatment involves determining the status of the granulocyte disorder in the subject. As used herein the “status” of a granulocyte disorder means the physiological stage or clinical condition of the cell, tissue, or the subject with the granulocyte disorder. It will be understood by those of ordinary skill that the status may reflect a number of different factors relating to the granulocyte disorder in the cell, tissue, or subject. These factors include, but are not limited to: the genotype of the cell, tissue, or subject, the genetic penetrance of the disorder, the length of time the disease has been manifested in the subject, and individual cell, tissue, and/or subject parameters that define the presentation of the granulocyte disorder in the cell, tissue, or subject. The status of the granulocyte disorder in a subject, cell, or tissue may change over time and thus the determination of a subject, cell, or tissue's status at a first time point may differ from the status of the cell, tissue, or subject's status at a second, subsequent time point.

The status of granulocyte disorder in a cell, tissue, or subject may be classified using general categories such as early-stage, mid-stage, or late-stage granulocyte disorder and the physiological manifestation of the granulocyte disorder may be generally classified as mild, medium, or severe, with various gradations in between. In some embodiments, the status of the disease means the level of pathogenesis from the disease. Thus, at early stages of a granulocyte disorder, pathogenesis may be mild or non-detectable and at mid and late stages the pathogenesis may be more pronounced. As used herein, the term “pathogenesis” means the clinical and physiological process and effects of the disease.

Accordingly, aspects of the invention may be used to treat any number of diseases including cancer, allergy, inflammation, infectious diseases, cardiovascular diseases, and other diseases associated with one or more leukocyte cell types (e.g., a mast cell and/or one or more granulocyte subtypes). Methods and compositions described herein in the context of mast cells and granulocytes may by applied to other leukocytes and leukocyte-selective markers that are described in the following examples and figures. For example, one or more CD4⁺ and/or CD8⁺ selective cell markers (e.g., with a value of 1.5 or more, 2 or more, 3 or more, 4 or more, 5 or more, in the tables included in the figures) may be used as targets to develop drugs that inactivate CD4⁺ and/or CD8⁺ cells and are thereby useful to promote allograft tolerance (e.g., tolerance of cell tissue and/or organ transplants) and/or tolerance of an implanted medical device or artificial structure). In addition, to enhancing transplantation tolerance, methods and compositions that modulate CD4⁺, CD8⁺ and/or other leukocytes, may be useful to treat cancer, infection, allergy, and other diseases described herein.

### EXAMPLE

In this study, we have used high density oligonucleotide probe array (GeneChip) to measure the expression levels of approximately 20,000 different transcripts in highly purified cells. These cells were basophils, eosinophils, neutrophils, monocytes (CD14⁺), T lymphocytes (CD4⁺ and CD8⁺ cells), B lymphocytes (CD19⁺), lung-derived mast cells, cord blood-derived cultured mast cells, and nasal polyp-derived fibroblasts. The GeneChip assay allows the simultaneous measurement of large numbers of transcripts using relatively small numbers of cells. Using this technology, we could even measure triplicate transcriptome levels of basophils, the most rare granulocytes in peripheral blood.

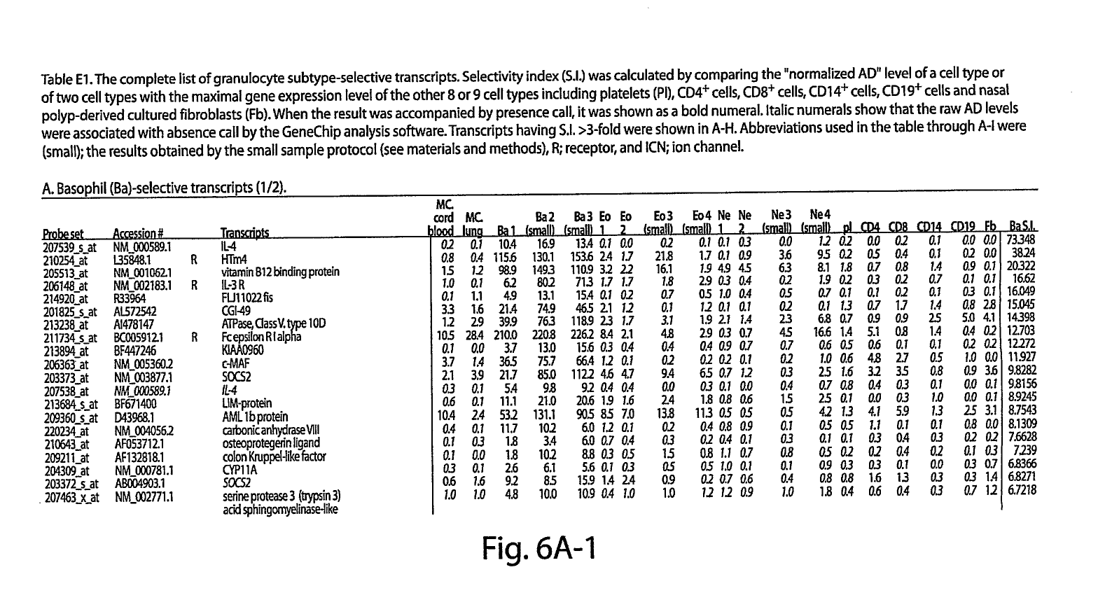

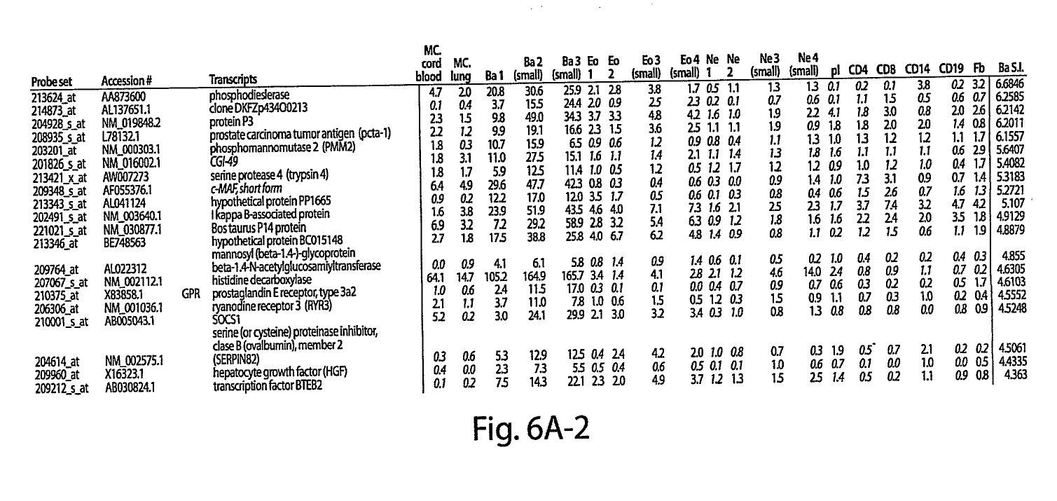

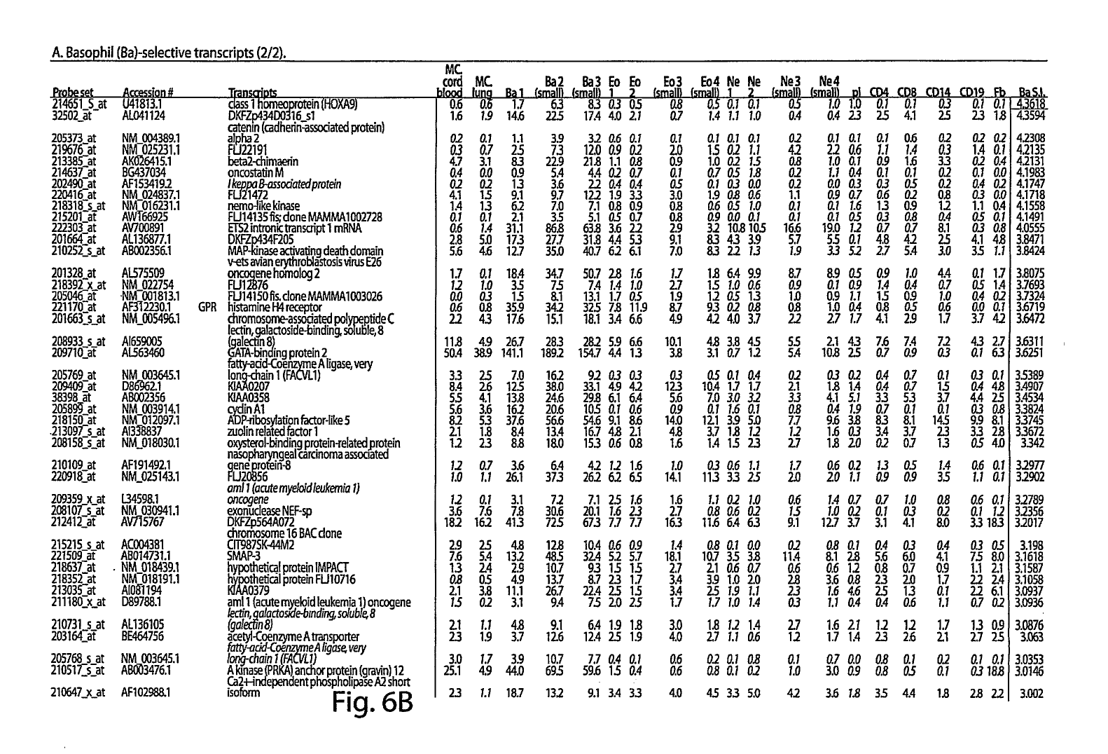

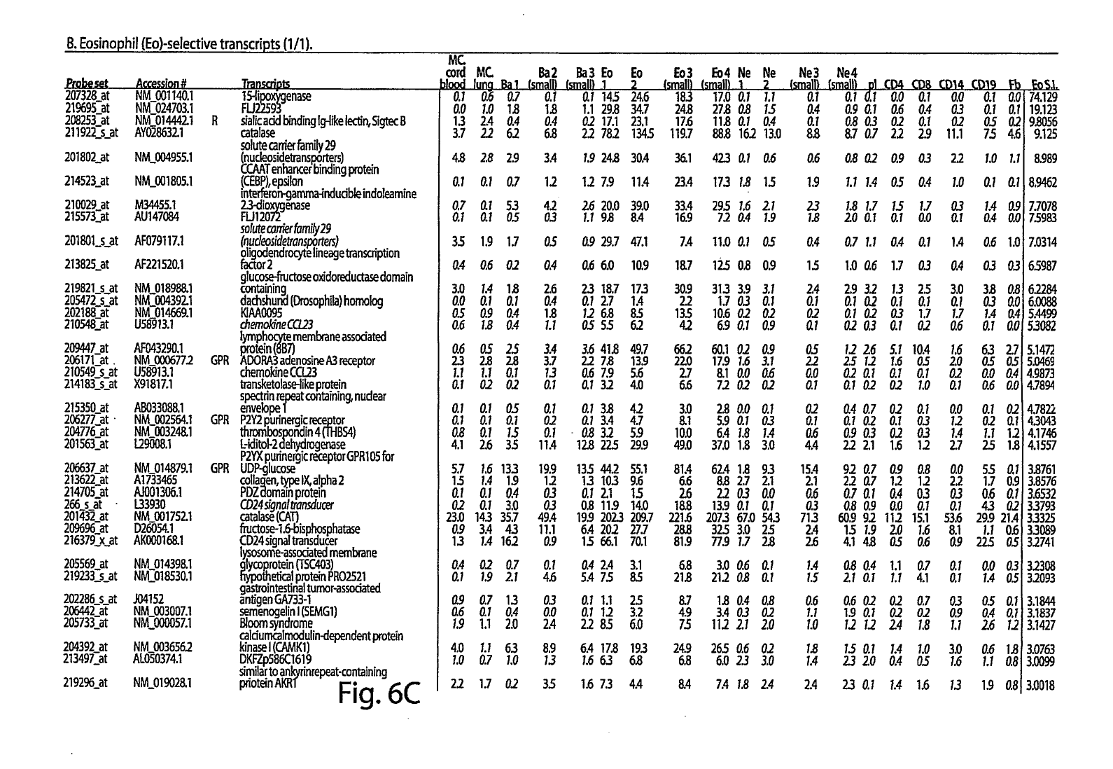

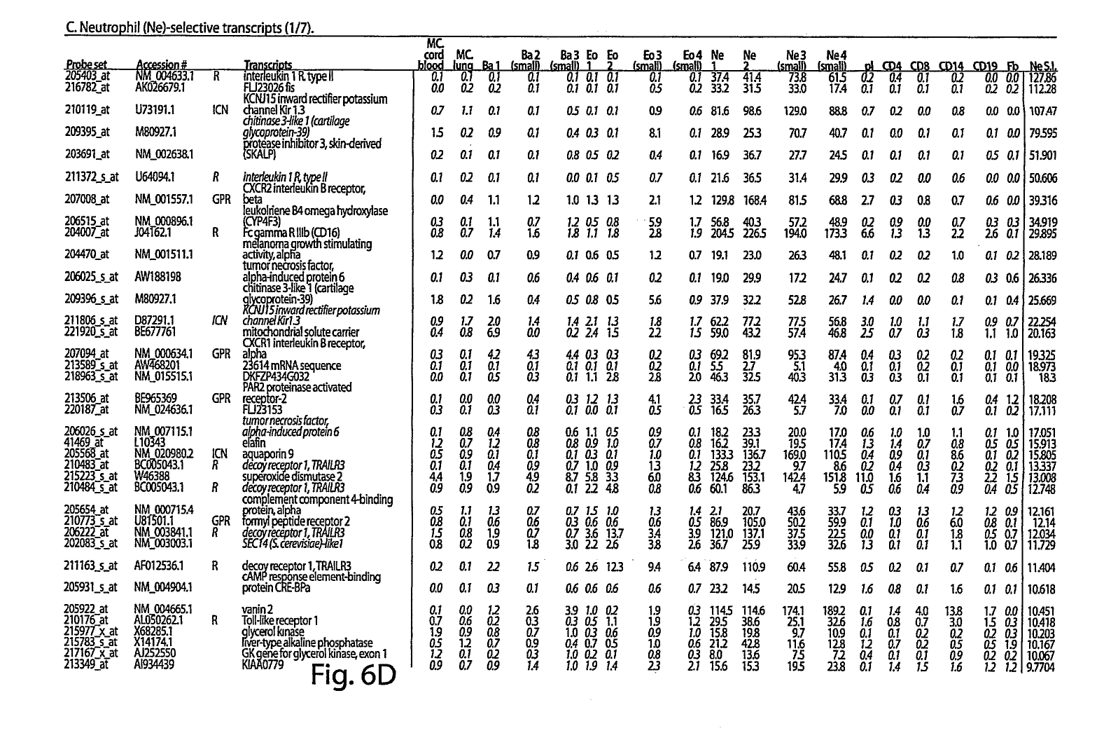

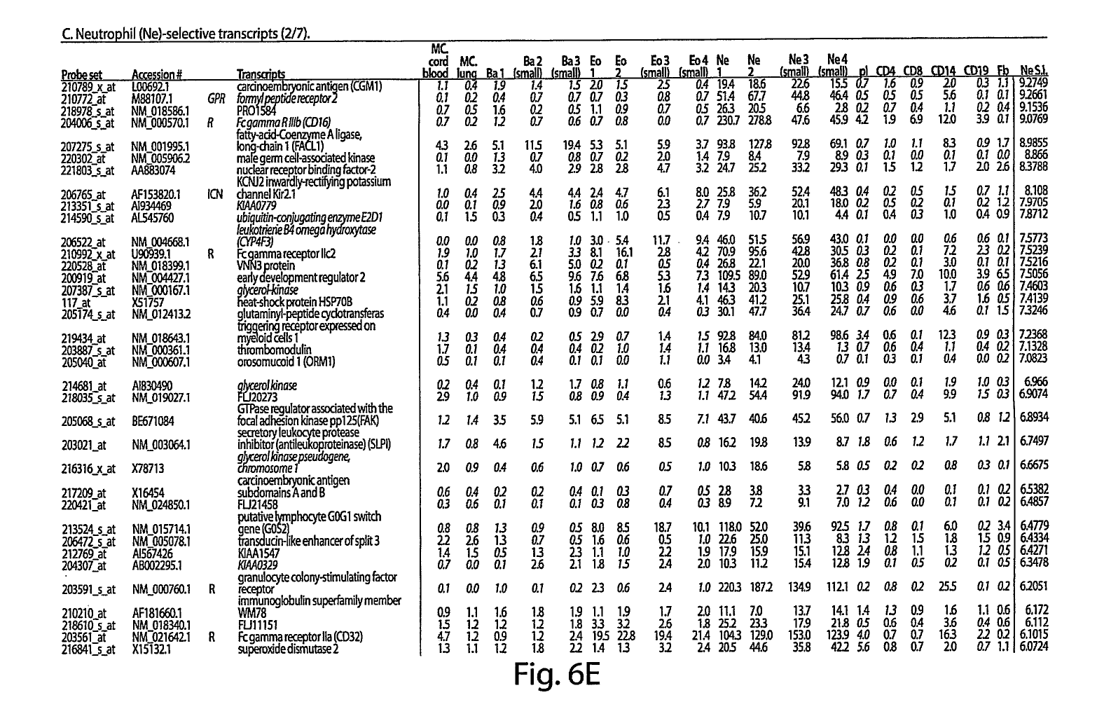

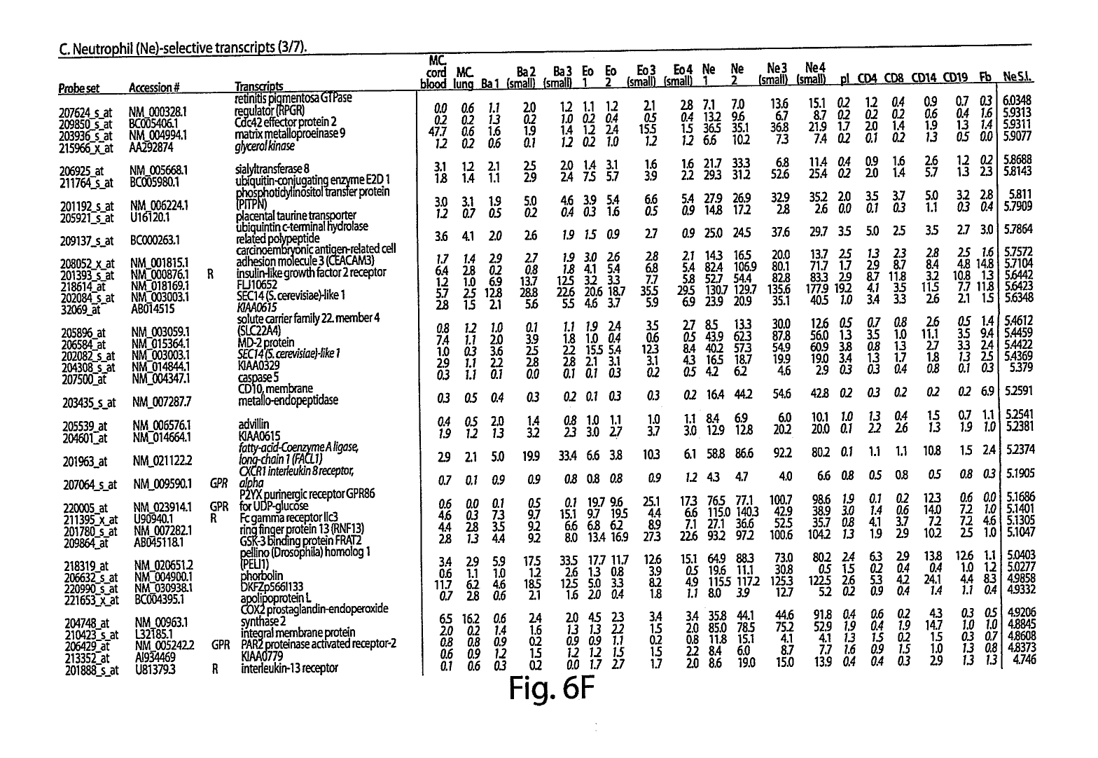

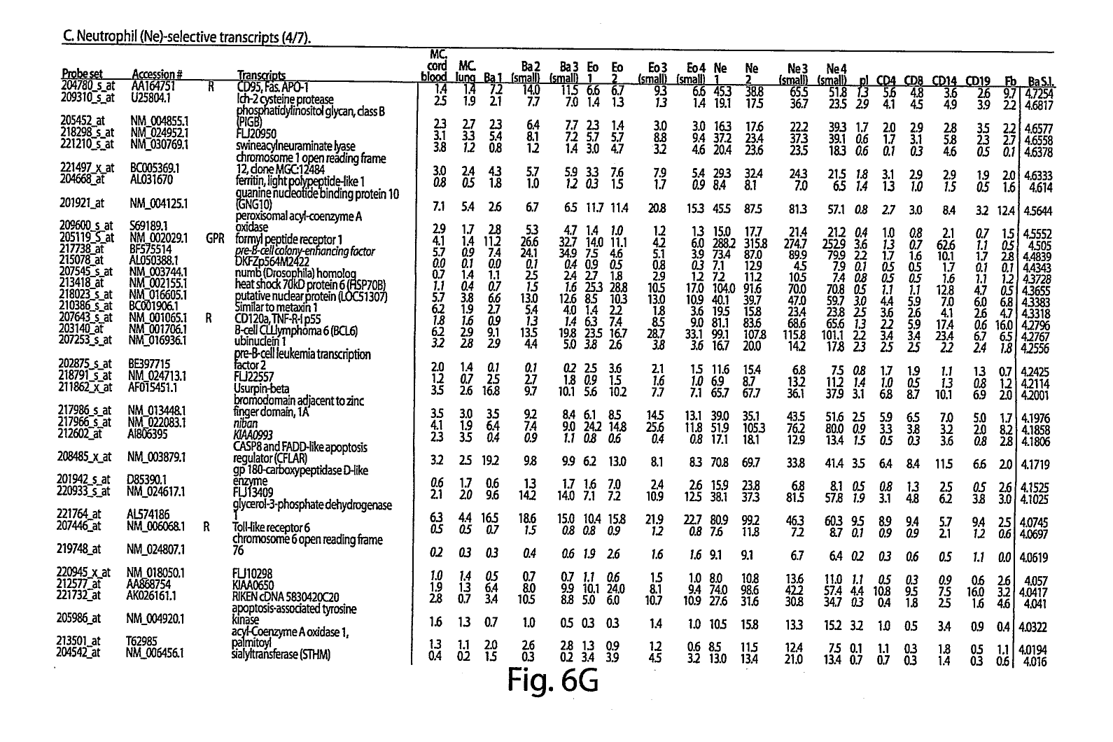

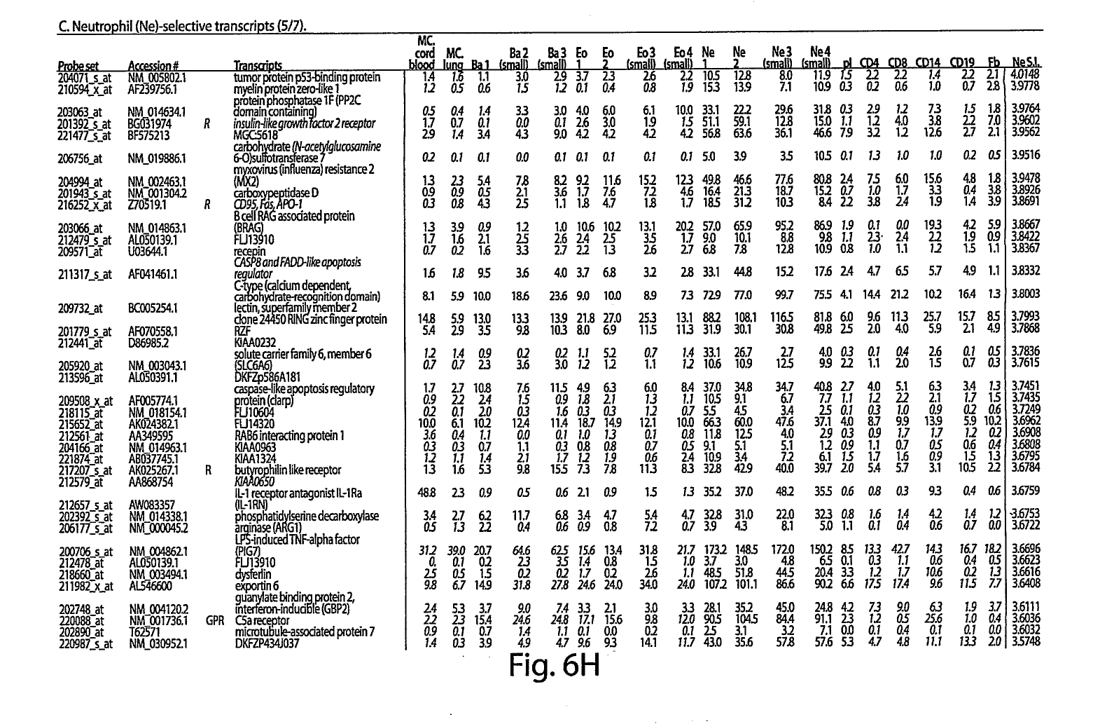

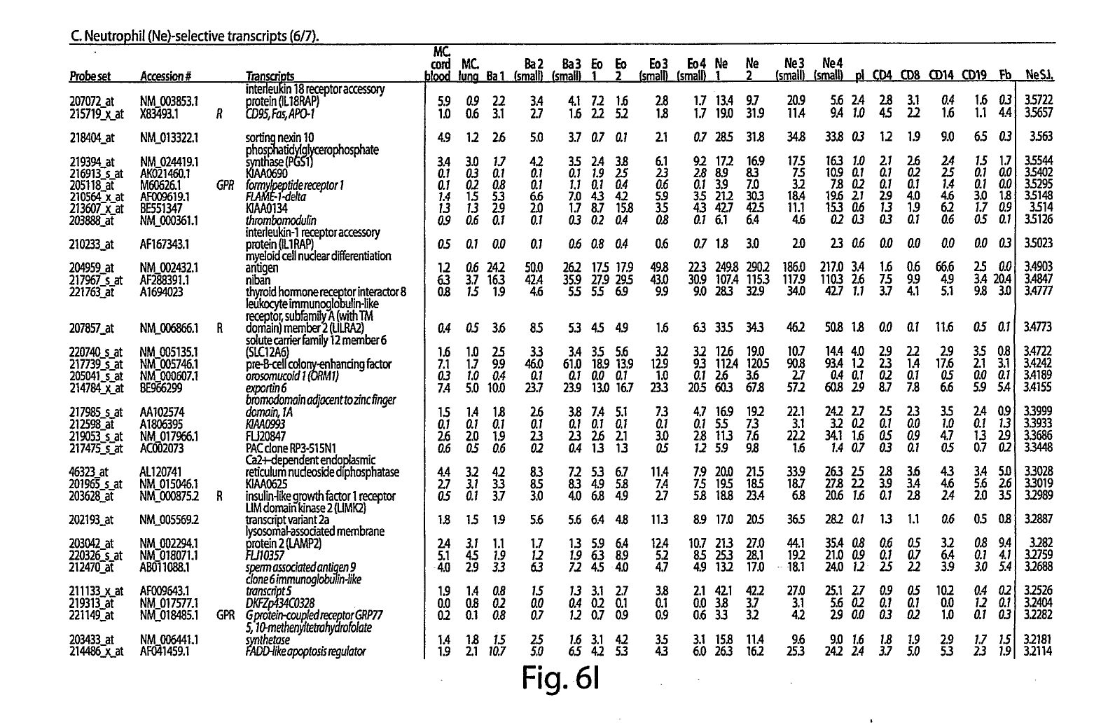

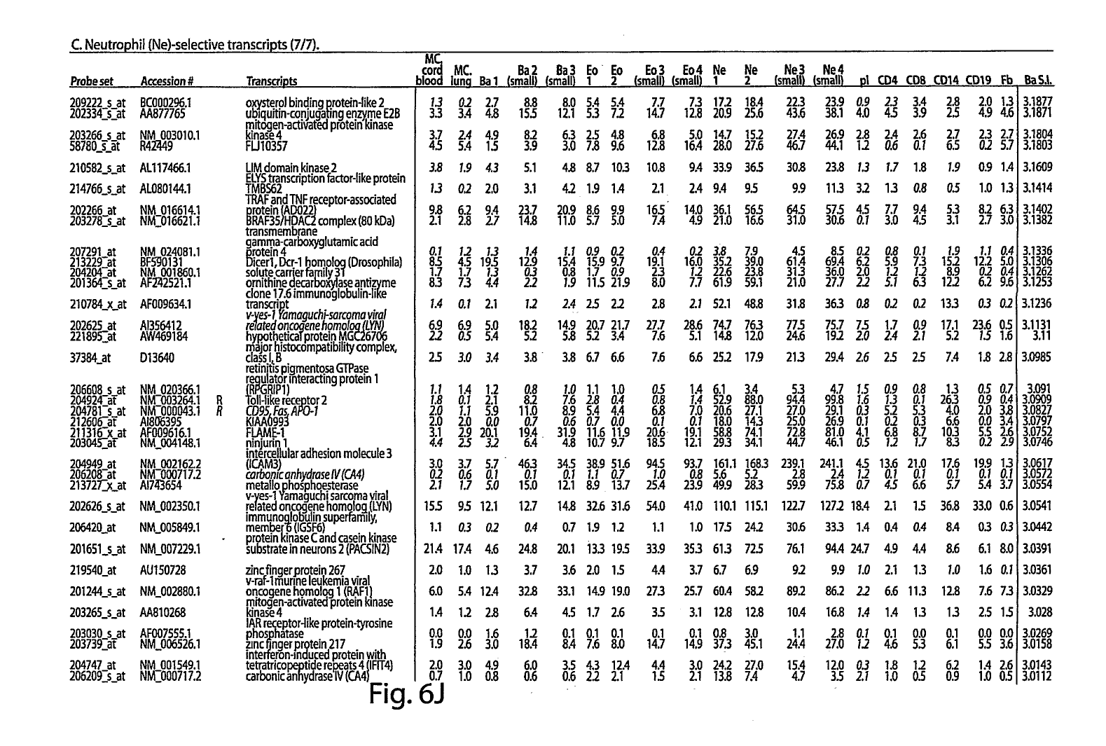

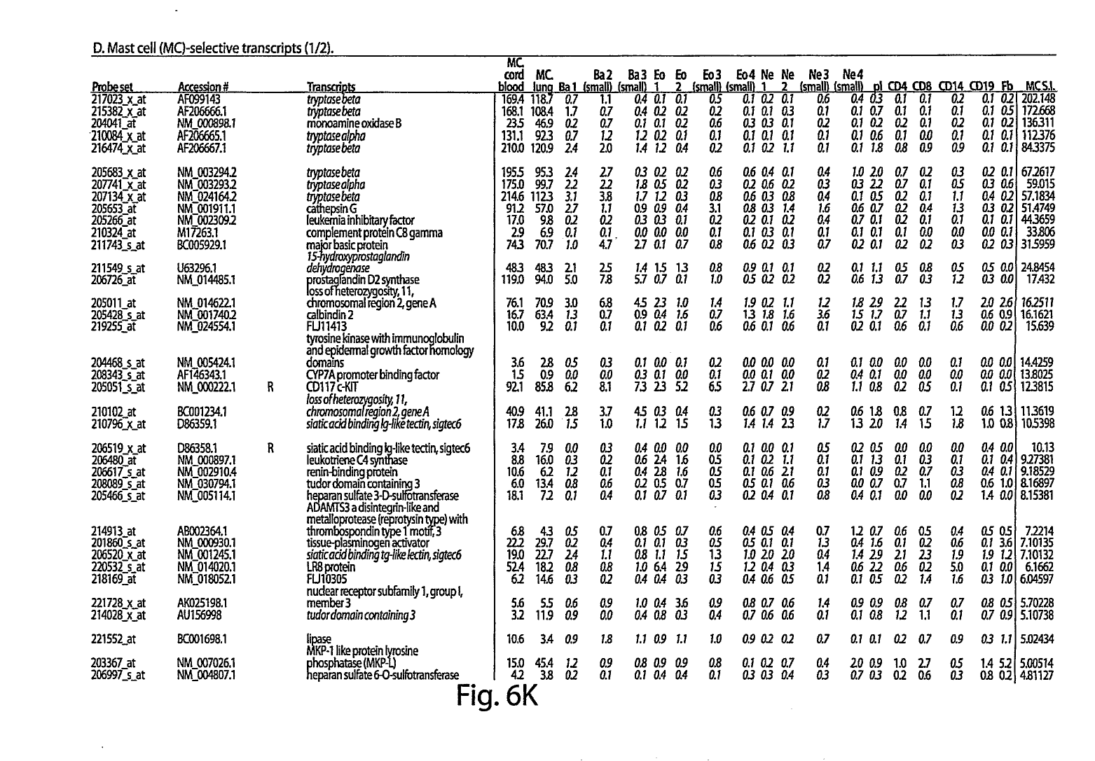

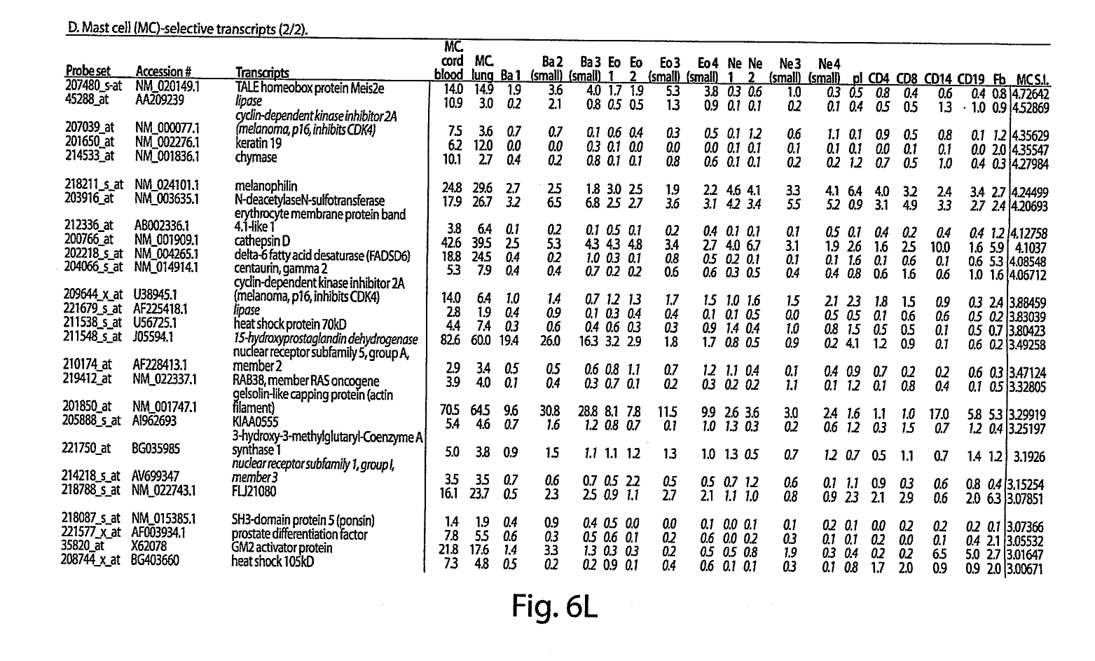

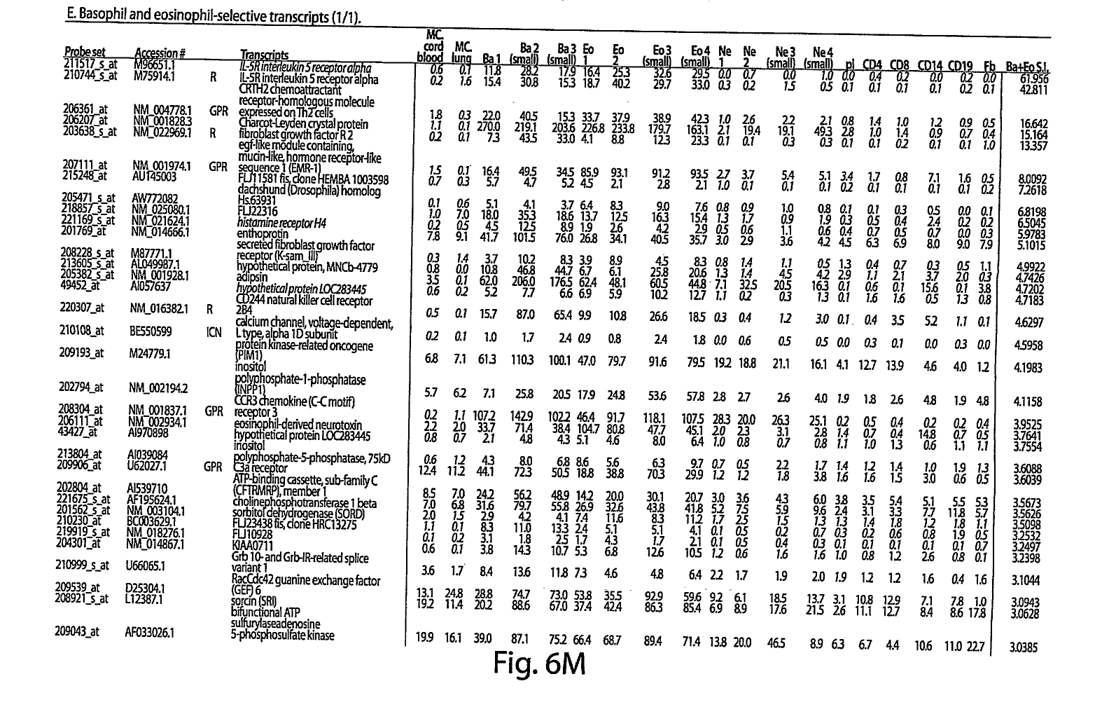

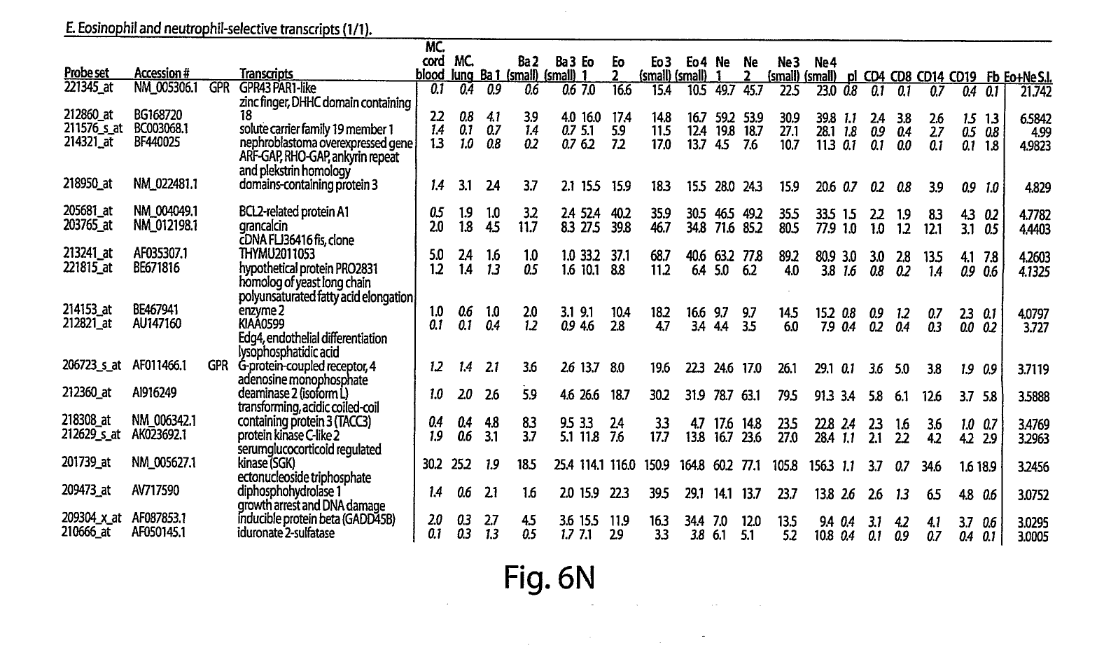

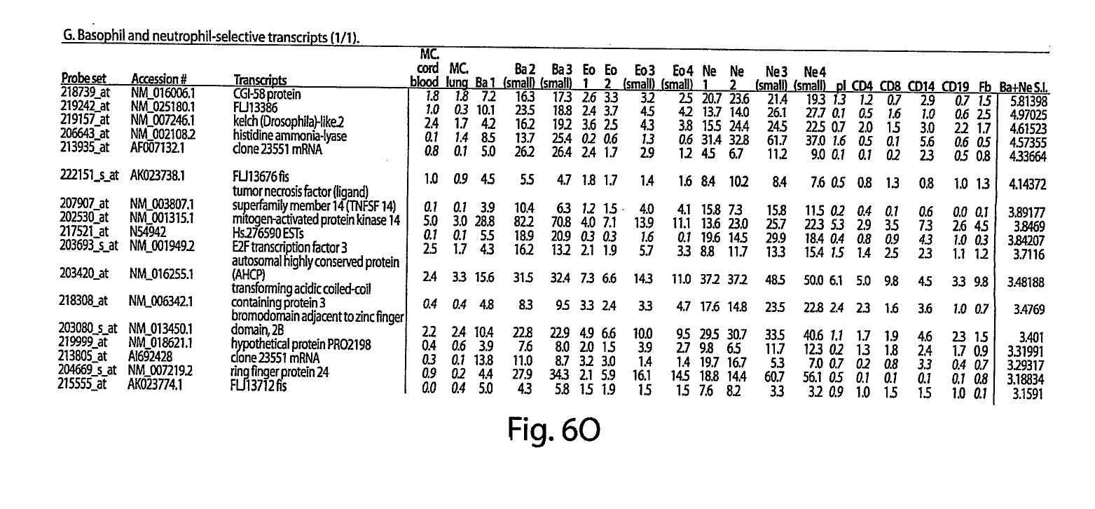

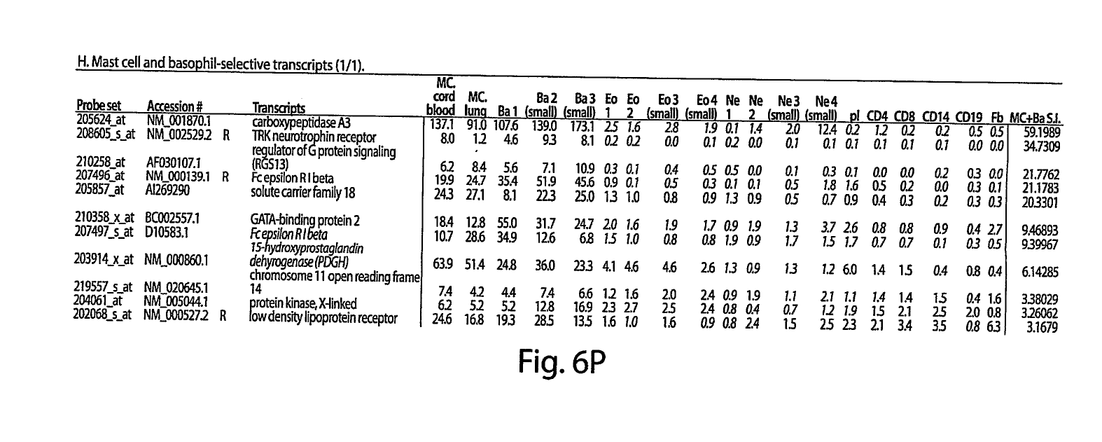

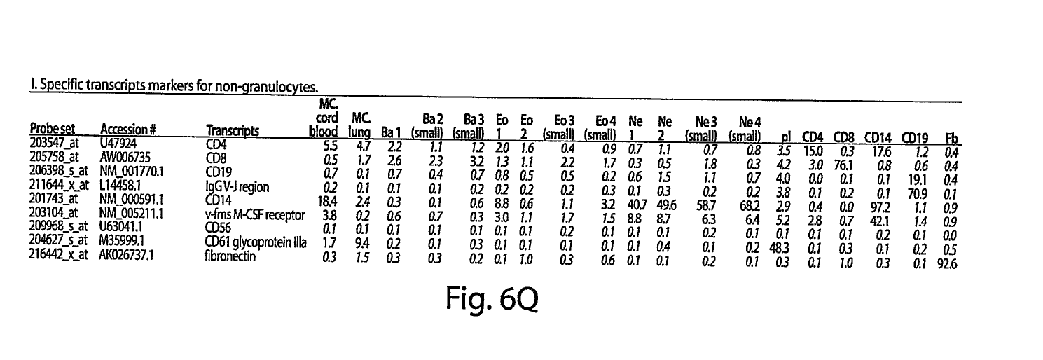

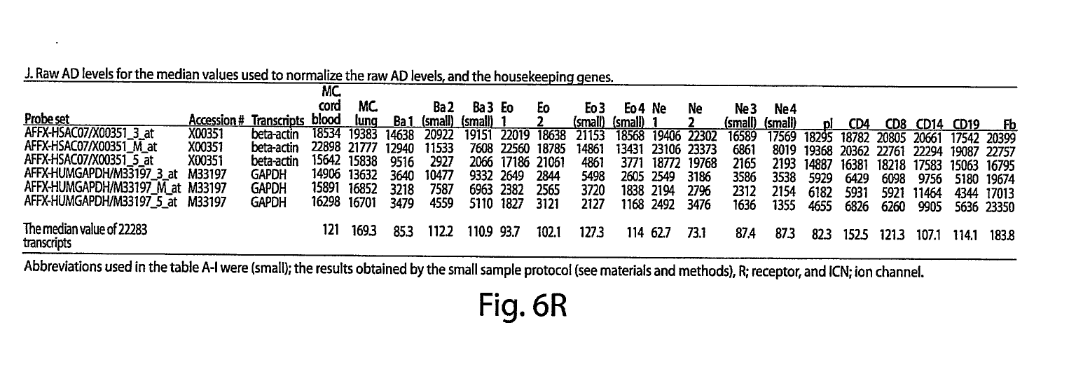

Cell type-selective transcripts were selected based on the following criteria; (1) the average “normalized AD” expression level of each gene in a certain cell type must be 3-fold or greater than the maximal level in other cell types, and (2) must be significantly (p<0.01) greater than that in other cell types. (3) The “AD” expression level provided with “absence” or “marginal” call by GeneChip Suite Software should be observed only once or not at all in the three or four independent experiments (3 experiments for basophils, 4 experiments for eisinophils and neutrophils) using different cell populations performed. (4) For the transcripts preferentially expressed for the two different cell types such as basophils and eosinophils, the average “normalized AD” expression levels in the two cell types should be within 3-fold of each other. Using these standards, we found 83 basophil-selective, 37 eosinophil-selective, 257 neutrophil-selective, 34 basophil-eosinophil-selective, 19 eosinophil-neutrophil-selective, and 17 basophil-neutrophil-selective transcripts. Due to the functional similarity with basophils, mast cell-selective transcripts were also examined, and 63 mast cell-selective and 11 mast cell-basophil-selective transcripts were also detected (Table 1, and FIGS. 6A-6R). Since mast cells, basophils and eosinophils play similar roles in allergic inflammation, the transcripts preferentially expressed for the three granulocytes by comparing their average “normalized AD” levels to other leukocytes. Thirty-four transcripts were then selected; however, most of them were overlapped with the transcripts listed in FIGS. 6A-6R. Only four transcripts (MYB, SAMSN1, BACE2, and CASP3) were found not overlapped, and they were not receptors or ion channels.

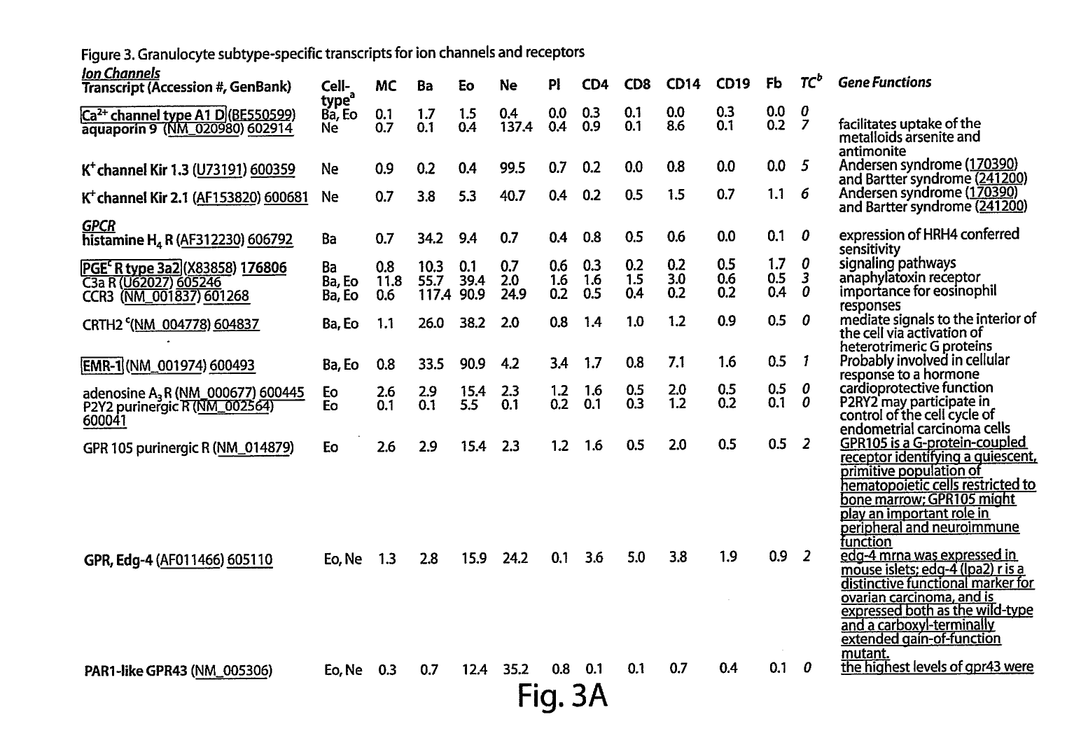

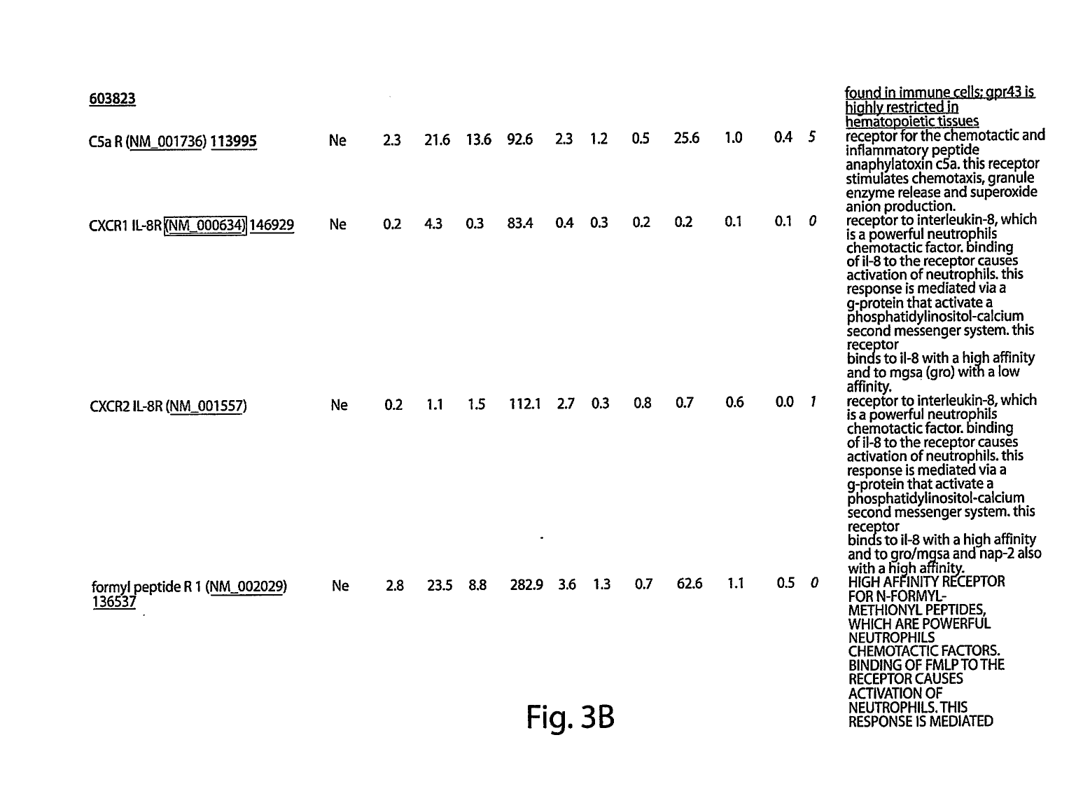

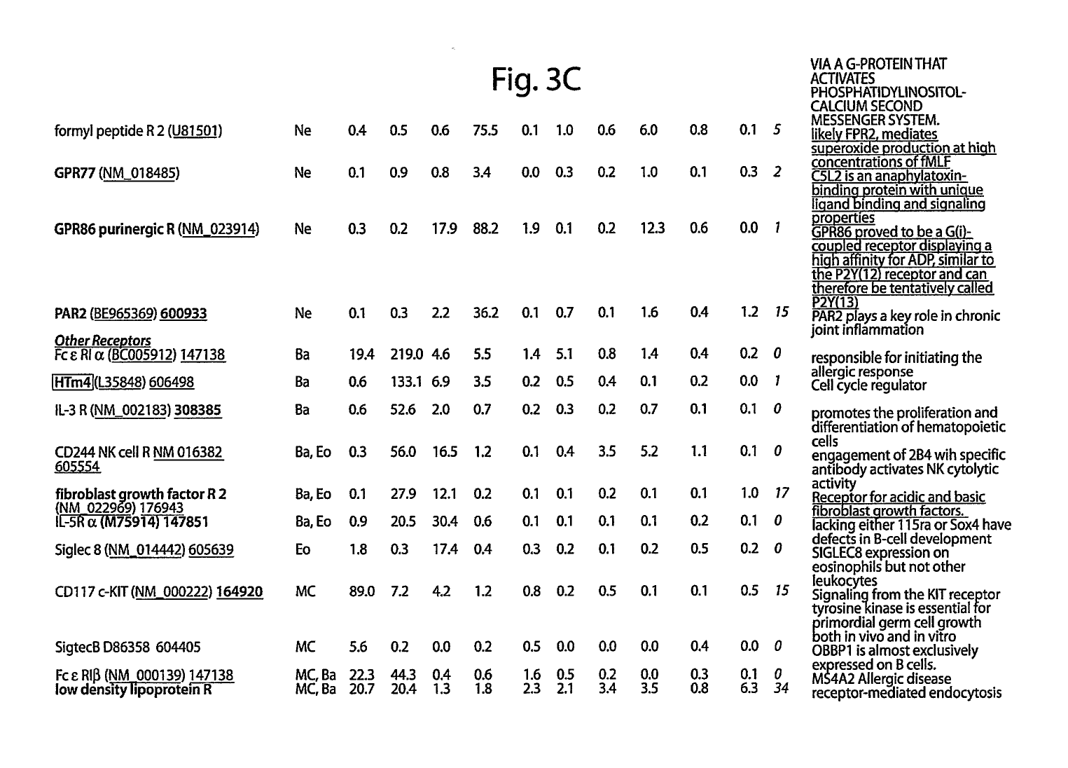

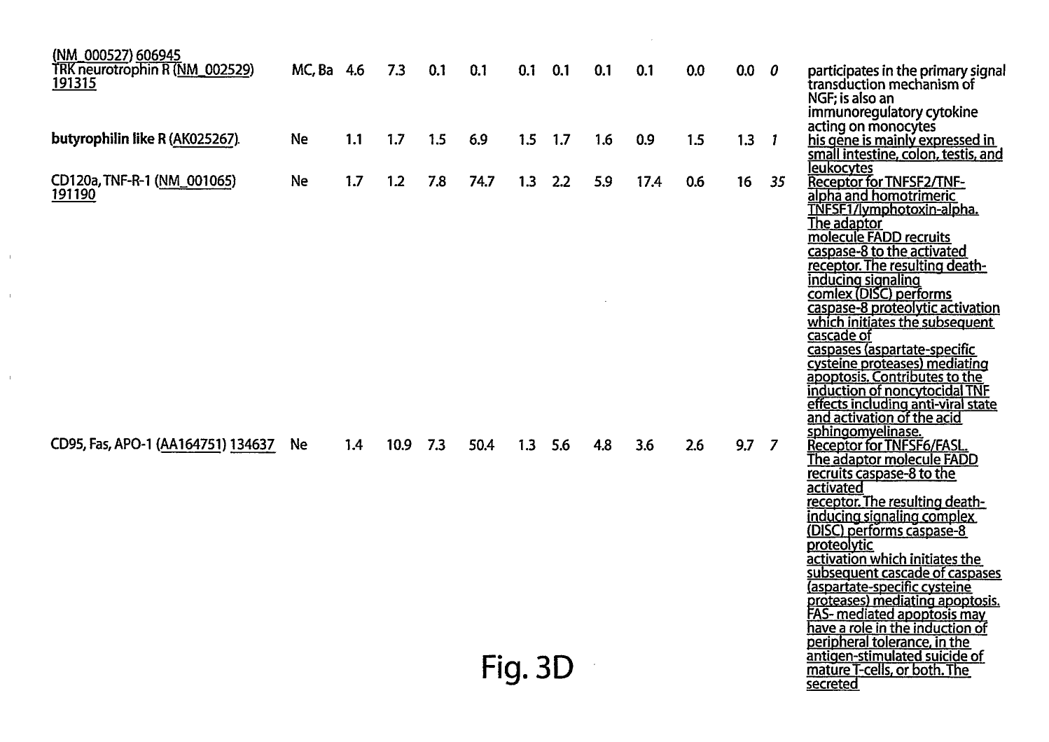

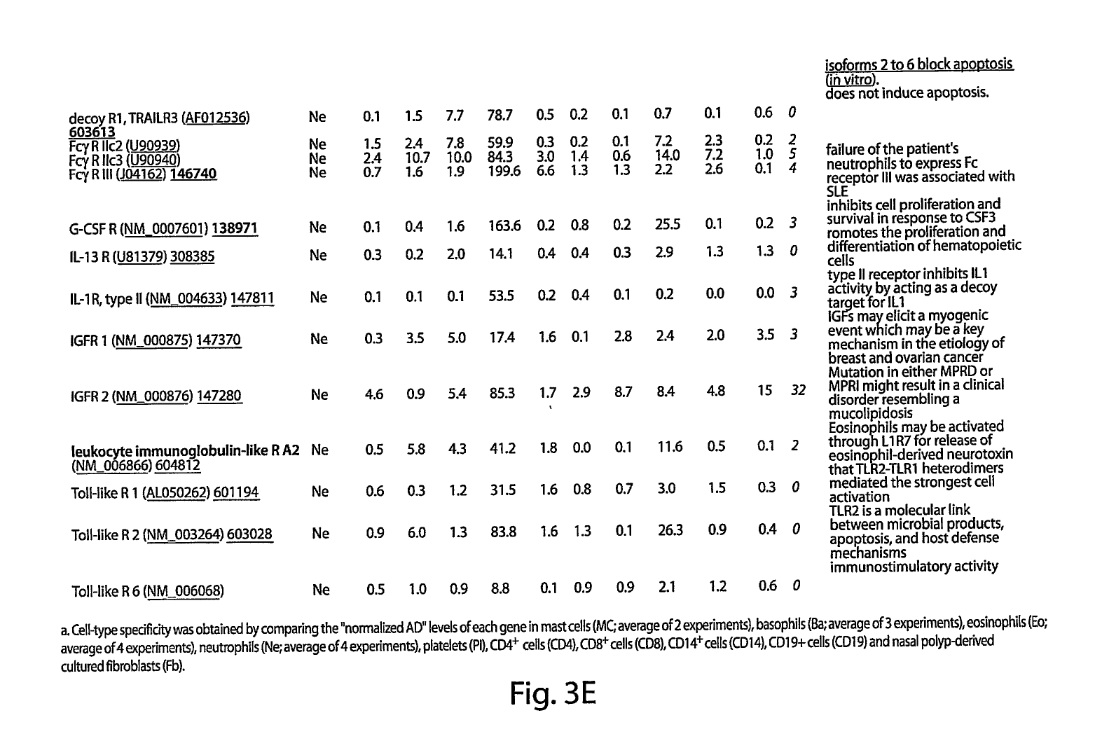

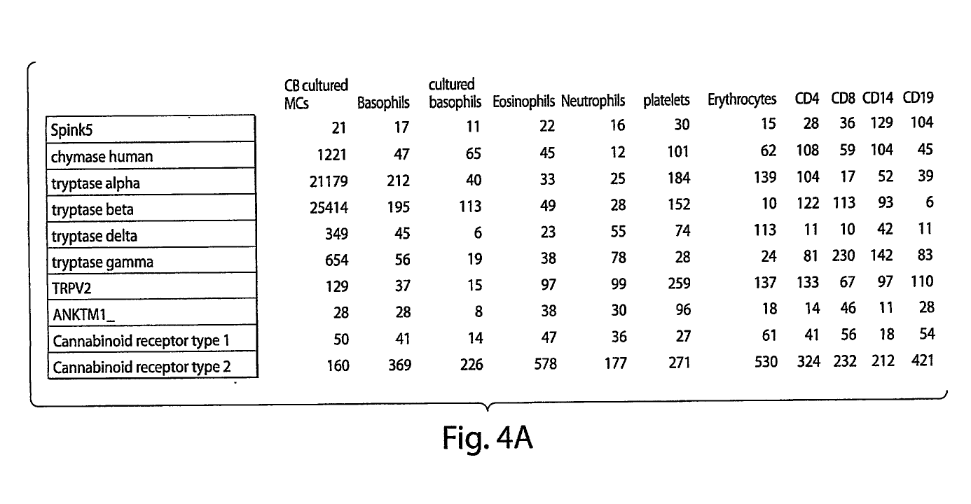

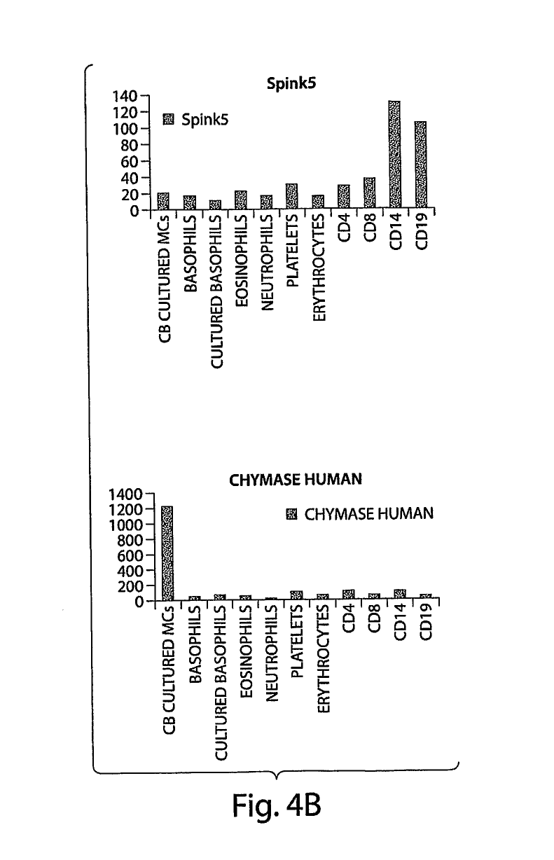

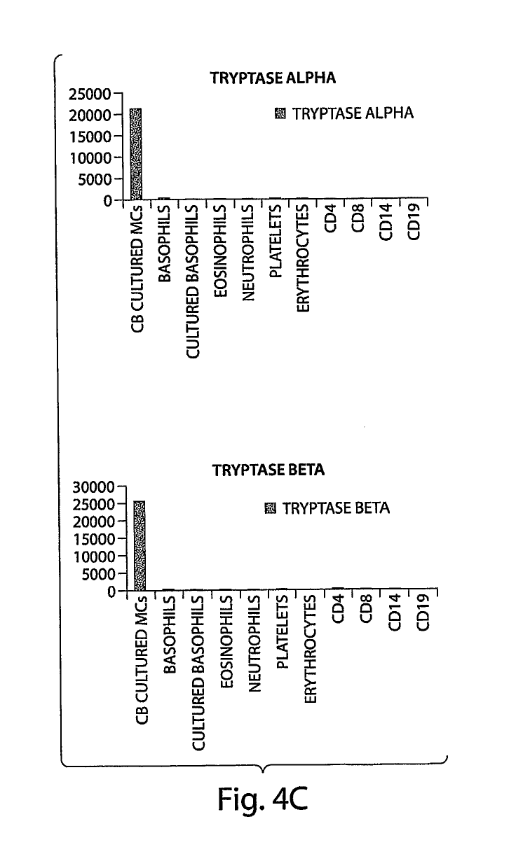

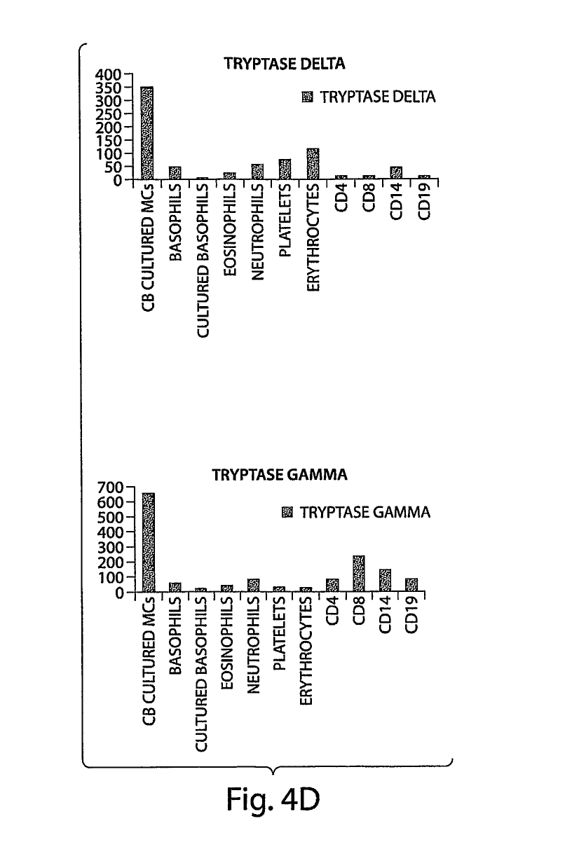

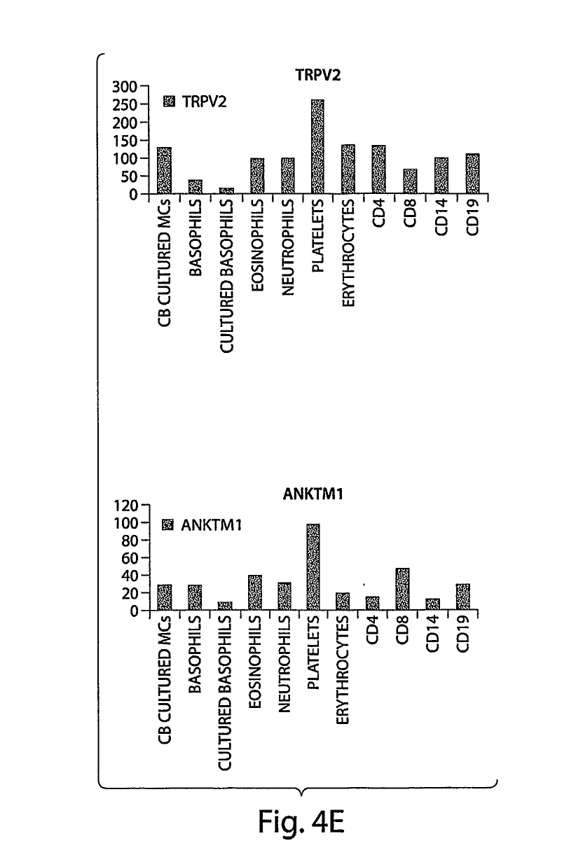

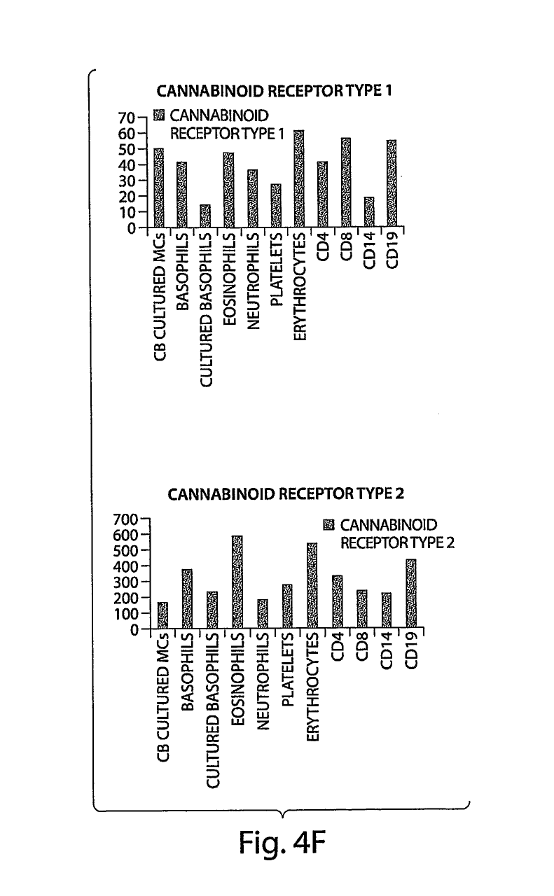

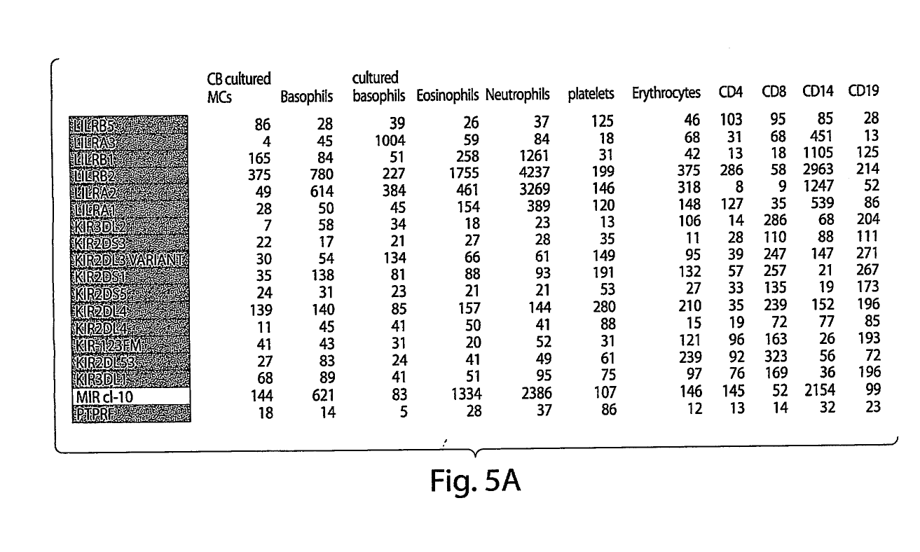

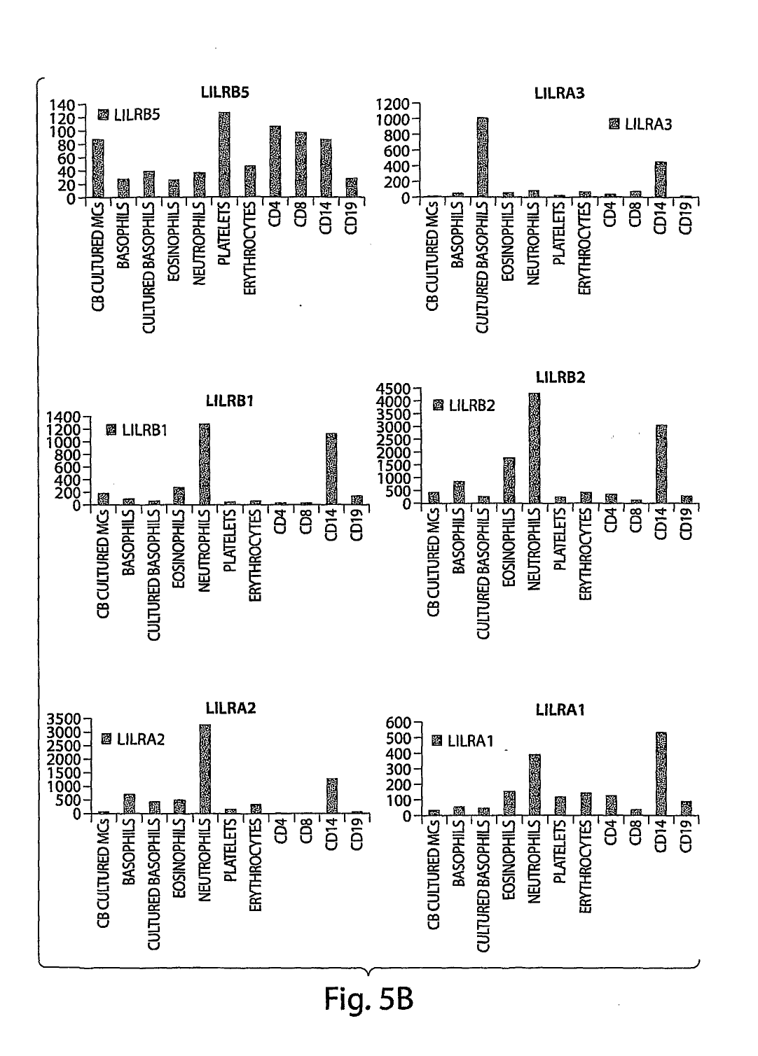

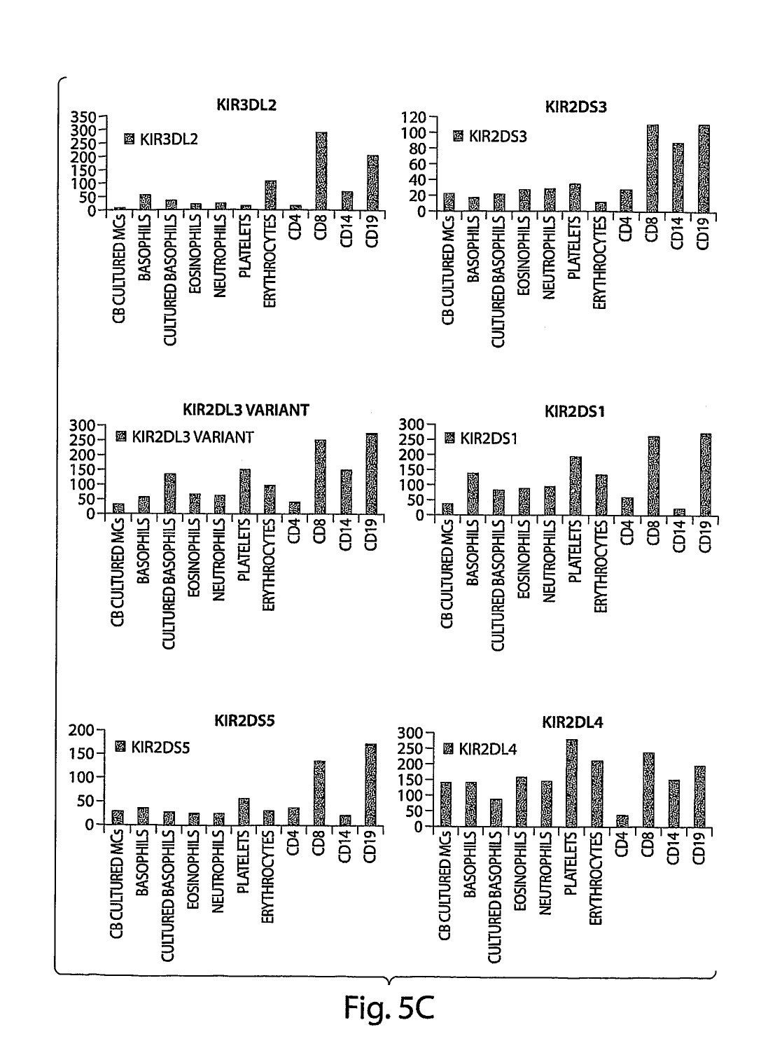

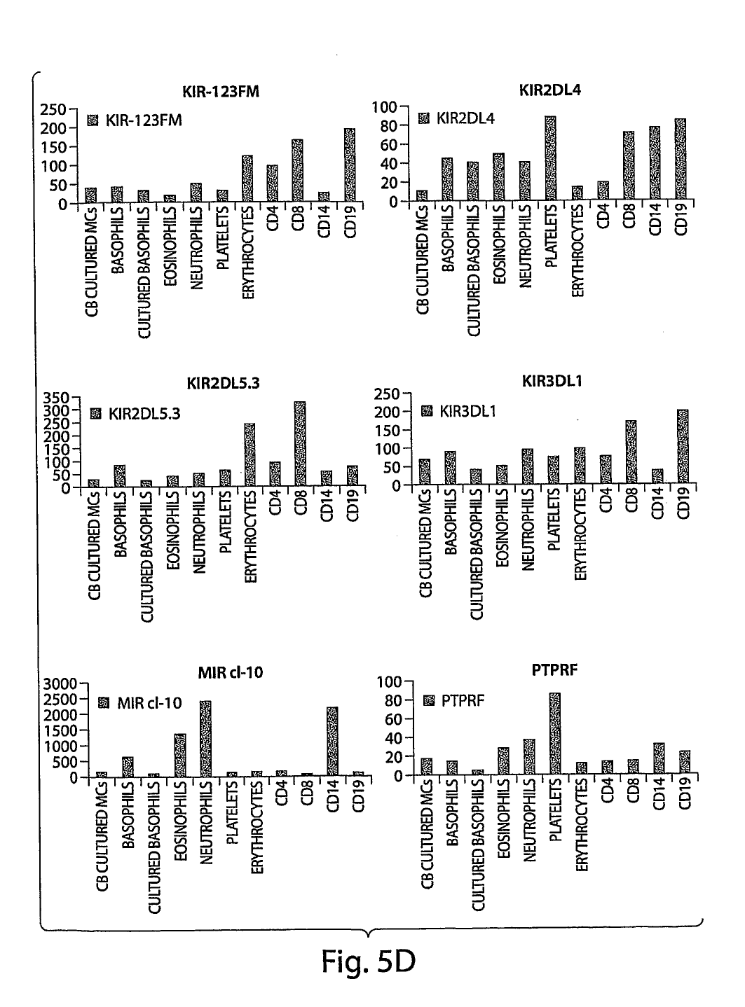

Among the 491 granulocyte-selective transcripts listed in FIGS. 6A-6R, 4 ion channels, 19 GPR and 28 other receptors were further selected (FIG. 3). When plural transcripts obtained by different probe sets had identical Genebank or Unigene accession numbers (http://www.ncbi.nlm.nih.gov/), the transcript showing the highest expression level was selected.

Ion Channels and Receptors Preferentially Expressed by Granulocytes other than Neutrophils

Eosinophils, basophils and mast cells play an important role in the pathogenesis of allergic diseases, but do not play an essential role in killing microbes except for nematodes. On the other hand, neutrophils play a crucial role in killing microbes such as bacteria. Caution should be taken in regulating neutrophil function even in the case of neutrophil-induced inflammation. Therefore, the molecules present only in granulocytes except for neutrophils would be important pharmaceutical targets for allergic disorders.^{1,2}

Among the 51 granulocyte-selective transcripts for ion channels and receptors, we identified 17 granulocyte-selective transcripts that have not been reported for their selective expression (shown in bold letters in FIG. 3). Of these 17 transcripts, eight were preferentially expressed by granulocytes other than neutrophils. Among these eight transcripts, the two transcripts for fibroblast growth factor receptor 2 and low density lipoprotein receptor were found to be expressed by multiple tissue cell types (shown at the Web site http://www.lsbm.org/index_e.html), which displays genomic expression of 55 different human tissue cells such as brain, heart and lung cells using the same experimental system. Affymetrix, U133A as ourts. Thus, they may not be suitable as a drug target because important organs that are unrelated to allergic inflammation (such as the brain) express it. Among the six novel transcripts found to be preferentially expressed by granulocytes except for neutrophils, we focus on the following four transcripts expressed by granulocytes including basophils. They were Ca²⁺ channel (CACNA1D), a prostaglandin E receptor, (EP3A2), epidermal growth factor-like module-containing mucin-like receptor (EMR) 1 (EMR1), and HTm4 (MS4A3).

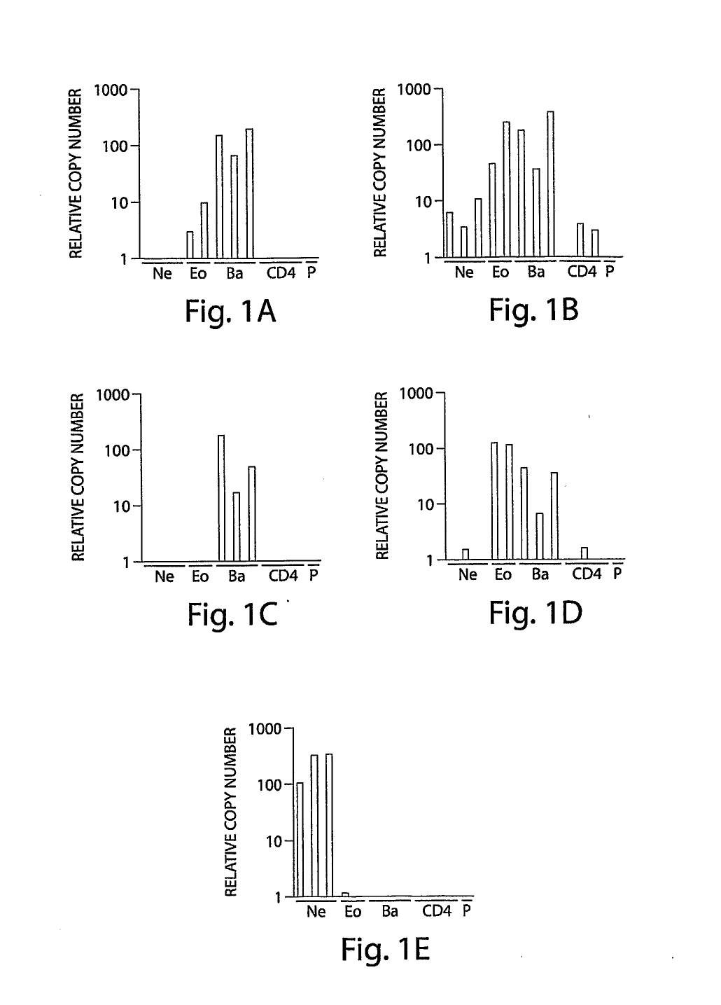

Basophils are the rarest granulocytes present in human peripheral blood and as such their complete transcriptional profiles remain unclear and no basophil-selective transcripts have previously been reported.²¹ Although eosinophils and mast cells have been considered as important therapeutic targets for allergic diseases for a long time, recent studies suggest the importance of basophils in pathogenesis of severe allergic diseases such as fatal asthma.^{2,22} Therefore, we further examined the selective expression of these four basophil-, or basophil-eosinophil-selective transcripts by using real-time RT-PCR. As shown in the FIG. 1, including a known molecule preferentially expressed by neutrophils, aquaporin 9,²³ the results obtained with GeneChip assay were confirmed by using this highly accurate and reproducible method.²⁴

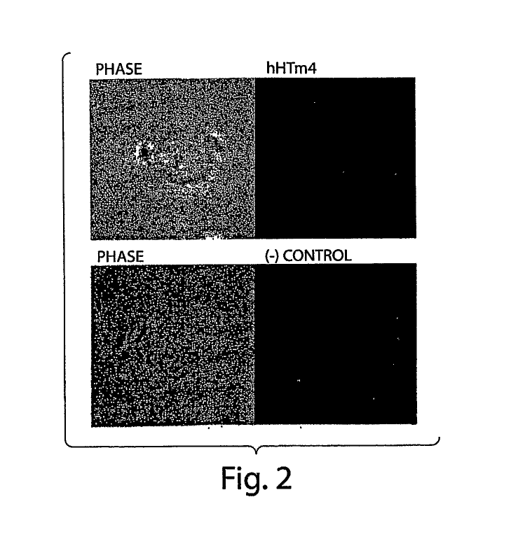

Among these four molecules, we could obtain a suitable antibody against HTm4, which is a member of a family of four transmembrane-proteins which include CD 20 and high affinity Fc receptor for IgE (FcεRI) β-chain.²⁵ Genetics provided evidence for the existence of multiple loci relevant to atopic asthma on chromosome 11q13, including HTm4.²⁶ Most recently, we have published data identifying HTm4 as a hematopoietic cell cycle regulator.²⁷ Using specific antibody against HTm4, we could detect the expression of HTm4 at the protein level in basophils (FIG. 2). To confirm whether these ion channels and receptors could be potential drug targets for diseases involving basophil activation, the amount of molecules expressed by various cell types should be quantified and the effect of any identified antagonists should be tested on the cell types found to express these molecules.

As has been well documented and expected, Fc ε RI β, IL-3 receptors, IL-5 receptors, chemokine receptor CCR3,^{1,2} sialic acid binding Ig-like lectin (Siglec)-8,²⁸ Siglec-6,¹⁵ histamine H4 receptor²⁹ and chemoattractant receptor-homologous molecule expressed on Th2 cells (CRTH2)³⁰ were preferentially expressed by basophils and/or eosinophils or mast cells. These consistent observations strengthened the reliability of the present methods and research strategy. Indeed, the antibody against Siglec-8 can induce selective apoptosis of eosinophils, and is expected to be useful therapeutically.²⁸

### Ion Channels and Receptors Preferentially Expressed by Granulocytes Including Neutrophils

Of the 17 transcripts that have not been reported as granulocyte-selective, nine were preferentially expressed by granulocytes including neutrophils. Pharmaceutical targets of selective granulocyte transcripts should treat inflammatory diseases without affecting the function of important organs that are unrelated to inflammation as well as the function of lymphocytes. However, four of the nine neutrophil-selective transcripts were expressed by multiple organ tissues. One of the four neutrophil-selective transcripts encoded proteinase-activated receptor (PAR)-2, a receptor for mast cell tryptase, which is linked to the pathogenesis of allergic diseases such as asthma.^{31,32} PAR-2 transcripts are also abundantly expressed by tissue types including skin and intestine which are often the target organs for allergic diseases. But the development of PAR-2 antagonists for use as anti-allergic drugs may be unsuitable because it may down regulate neutrophil function and thereby induce bacterial infection.

### General Discussion

We identified 51 granulocyte-selective genes for ion channels and receptors by examining approximately 20,000 kinds of transcripts derived from 16,000 genes from 10 different types of cells using U133A GeneChip, which covers approximately half of the genes present in the human genome. The majority of these transcripts encoded molecules known or expected to be granulocyte subtype-selective such as the IL-3 receptor and Fc ε receptors.

Mast cells expressed low levels of Fc ε RI α compared to basophils, and that even neutrophils expressed a substantial level of the receptor (FIG. 3). This raises the possibility that GeneChip assay may not be suitable for detecting selective molecules. In the present study, however, only the GeneChip data obtained using cord blood-derived mast cells and lung mast cells could be employed due to the strict data selection based on the RNA quality (see Methods). As has been reported,^{14,33} peripheral blood-derived cultured mast cells or skin-derived mast cells expressed approximately 10-fold Fc ε RI α mRNA compared to cord blood-derived mast cells (data not shown). Also, as shown in FIGS. 6A-6R, only 2 of the 4 neutrophil samples expressed Fc ε RI α mRNA. This may be explained by the observation that only neutrophils obtained from some allergic donors express the molecule.³⁴

We unexpectedly found 17 granulocyte-selective transcripts including HTm4. Basophil- and/or eosinophil-selective transcripts identified in our study could be potential therapeutic targets for allergic diseases because these granulocytes play a crucial role in allergic inflammation.^{1,2} Granulocyte-selective transcripts could also be drug targets for other inflammatory diseases such as systemic vasculitis.^{3,4} Analysis of cell type-selective transcripts from database searches is expected to minimize the efforts required for drug discovery. The public database (http)://www.lsbm.org/index_e.html) shows that some granulocyte-selective transcripts (18 out of 51) detected in our study are abundantly expressed by multiple (more than 3) organ tissue cell types using the same GeneChip U133A probe array. Thus, the safety of any candidate drug must be evaluated by comparing its efficacy (on granulocytes) with its toxicity (to organs). Six out of the 17 novel granulocyte-selective molecules may be excluded from drug development due to their expression in multiple organs unrelated to the diseases. Thus, our approach has identified 11 receptors and ion channels with therapeutic potential. Especially, among the 11 receptors and ion channels, seven were basophil- and/or eosinophil-selective and were not expressed by other organs, indicating that they may be potential targets for anti-allergic drugs.

Finally, it should be stressed that basophils, the rarest leukocytes, have recently been found to play a more crucial role than we ever proposed in the pathogenesis of intractable allergic diseases such as fatal asthma.^{35,36} Thus, targeting basophil receptors and ion channels such as HTm4 and Ca²⁺ channel CACNA1D is particularly expected for the future drug discovery. The importance of molecules known to be expressed by basophils may be reevaluated regarding its selectivity. Freshly-isolated resting basophils expressed the highest level of IL-4 compared to other cell types. Because the basophil purification procedure requires more isolation steps, ex vivo manipulation may activate the cells. However, it should also be noted that basophils have been recently found as the major source of IL-4 at least in asthma models.^{37,38}

### Materials and Methods

### Purification of Leukocytes

All human subjects in this study provided written, informed consent, and the Ethical Review Boards at the relevant hospitals (National Center for Child Health and Development, and Jikei University School of Medicine) approved the study. The subjects used in this study were all healthy volunteers, especially having no allergic diseases.

Granulocytes and mononuclear cells were separated from venous blood of normal volunteers. Human basophils were semipurified by means of Percoll (Pharmacia, Uppsala, Sweden) density gradient centrifugation, and the cells were further purified by negative selection through use of a MACS Basophil Isolation Kit (Miltenyi BioTech, Bergisch-Gladbach, Germany), as described previously.¹⁷ Eosinophils were isolated by using Percoll (1.090 g/mL) density centrifugation. The eosinophils were further purified by negative selection with anti-CD16-bound micromagnetic beads, as described previously.¹⁸ Neutrophils were isolated by using Percoll (1.085 g/mL) density centrifugation and further purified by negative selection using anti-CD81 antibody and antimouse IgG-bound micromagnetic beads to eliminate contaminating eosinophils. These granulocytes purified from human peripheral blood were spun down onto slide glass by Cytospin II (Shandon Southern Instruments, Inc., Sewickley, Pa.). The purity of these cells was evaluated based on 500 cells stained with May-Grünwald and Giemsa solutions.

For preparation of lymphocytes and monocytes, peripheral blood mononuclear cells (PBMNC) were isolated by centrifugation on lymphocyte separation medium (Organon Teknika Corp., Durham, N.C.). Monocytes (CD14⁺ cells) were prepared using magnetic beads-conjugated CD14⁺ antibody (CD14 MicroBeads; Miltenyi Biotec) from PBMNC. CD4⁺ and CD8⁺ cells were also respectively sorted using magnetic beads-conjugated CD4⁺ (CD4 MicroBeads; Miltenyi Biotec) and CD8⁺ antibodies (CD8 MicroBeads; Miltenyi Biotec) from PBMNC after depletion of CD14⁺ cells with MACS CD14 MicroBeads (Miltenyi Biotec). The purity of CD4⁺, CD8⁺ and CD14⁺ cells was evaluated by staining the magnetic beads—conjugated cells compared to feasible control cell preparations such as unpurified cells with FITC-labeled goat anti-mouse Immunoglobulin (BD Pharmingen, Tokyo, Japan). Peripheral B cells were purified by a combination of negative (MicroBeads—conjugated antibodies to CD3, CD7, CD14, CD42b, and CD56; Miltenyi Biotec) and positive (CD19 MicroBeads; Miltenyi Biotec) selection using MicroBeads (Miltenyi Biotec). To obtain platelet rich plasma, blood samples were mixed with 3.8% (w/v) sodium citrate solution (9:1) and centrifuged at 260×g for 15 min. at 20° C. To remove any contaminating erythrocytes and leukocytes, the plasma was centrifuged again at 260×g for 15 min.

Human mast cells were derived from cord blood CD34⁺ progenitor cells as described previously.¹¹⁻¹⁴ Briefly, progenitor cells purified from peripheral blood by CD34⁺ isolation kits (Miltenyi Biotec), were cultured in Iscove's modified Dulbecco medium supplemented with 1% insulin-transferrin-selenium supplements (Life Technologies), 50 μM 2-mercaptoethanol, antibiotics, and 2% fetal calf serum in the presence of 100 ng/ml stem cell factor and 50 ng/ml IL-6. After 11 to 14 weeks of culture, tryptase positive cells represented more than 99% of the cells.

### Purification of Human Lung Mast Cells and Nasal Polyp-derived Fibroblasts

Normal human lung tissue dissected during surgery was obtained macroscopically after informed consent. Human lung mast cells were dispersed from chopped lung specimens by an enzymatic procedure and were purified by magnetic bead affinity selection using the mAb anti-kit, YB5.B8 (BD PharMingen, San Diego, Calif.) as described previously.¹⁹ The cells were further cultured in the presence of SCF and interleukin 6 (IL-6) for several weeks. Human nasal polyp-derived fibroblasts were obtained as previously reported.²⁰

### GeneChip Expression Analysis

Human genome-wide gene expression was examined using the Human Genome U133A probe array (GeneChip, Affymetrix, Santa Clara, Calif.), which contains the oligonucleotide probe set for 22,000 full-length genes. Experiments were performed in accordance with the manufacturer's protocol (Expression Analysis Technical Manual) and previous reports.¹¹⁻¹⁴ Total RNA (3-10 μg) was extracted from 10⁷ cells. Double-stranded cDNA was synthesized using a SuperScript Choice system (Life Technologies) and a T7-(dT)24 primer (Amersham Pharmacia Biotech, Buckinghamshire, UK). The cDNA was subjected to in vitro transcription in the presence of biotinylated nucleoside triphosphates using a BioArray high-yield RNA transcript labeling kit (Enzo Diagnostics, Farmingdale, N.Y.). The biotinylated cRNA was hybridized with a probe array for 16 h at 45° C. In some experiments as indicated in the supplementary table, biotinylated cRNA was prepared using two-cycles of cDNA synthesis and in vitro transcription for target amplification was performed according to the manufacturer's “The Small Sample Labeling Protocol version II” (Affymetrix, Inc). For the latter protocol, we employed 100 ng total RNA. After washing, the hybridized, biotinylated cRNA was stained with streptavidin-phycoerythrin (Molecular Probes, Eugene, Oreg.) and then scanned with a HP gene array scanner. The fluorescence intensity of each probe was quantified using a computer program, GeneChip Analysis Suite 5.0 (Affymetrix). The expression level of single mRNA was determined as the average fluorescence intensity among the intensities obtained by 11 paired (perfect-matched and single nucleotide-mismatched) probes. If the intensities of mismatched probes were very high, gene expression was judged to be absent, even if a high average fluorescence was obtained with the GeneChip Analysis Suite 5.0 program. The level of gene expression was determined as the average difference (AD) using the GeneChip software. Each AD level was then normalized by dividing it with the median value of 22,283 AD levels obtained in an experiment (“normalized AD” level).

### Real-Time Reverse Transcriptase (RT)-PCR

Total RNA was isolated using Isogen (Nippon gene, Tokyo, Japan) according to the manufacturer's instructions and quantified by measuring the absorbance at 260 nm. RNA was subsequently treated with DNase I (Life Technologies) reverse transcribed using Superscript II reverse transcriptase (Life Technologies). Real-time RT-PCR was performed 10 ng cDNA in 25 μl of final volume using the primers and probes supplied by “Assays-on-Demand Gene Expression system” (PE Applied Biosystem) according to the manufacturer's instructions. Measurement of gene expression was performed using the ABI PRISM 7700 Sequence Detector. The expression level of each gene was normalized to a GAPDH.

Staining of Basophils with Anti-HTm4

Basophils purified from human peripheral blood with Basophil Isolation Kit (Miltenyi Biotec) were spun down onto slide glass by Cytospin II (Shandon Southern Instruments Inc., Sewickley, Pa.). Cells were fixed with aceton for 1 minute and then blocked in goat serum in 50 mm TRS-Cl, pH 7.4 for 1 hour. Cells were further incubated for 2 hours with 2 μg/ml of the polyclonal antibody rabbit anti-hHTm4. Cells were then washed three times with PBS and incubated with a secondary antibody, highly cross-adsorbed Alexa Fluor® 546-conjugated goat anti-rabbit IgG (H+L) (Red) (Molecular Probes, Eugene, Oreg.) for one hour. After three PBS washes, air dried cells were further mounted using the Prolong Anti-Fade Kit (Molecular Probes, Eugene, Oreg.). Slides were scanned by Zeiss Laser Scanning Microscope 5 Pascal (Carl Zeiss Microimaging Inc, Thornwood, N.Y.).

### Purity and Viability of the Leukocytes, and RNA Quality

We used leukocyte samples in this study only if the purity of each cell type was at least 98%, but there are >0.5% contaminated cells in any of the samples. We could not evaluate the purity of CD19⁺ cells and platelets due to lack of feasible controls or methods. However, specific transcript markers for non-granulocytes (CD4, CD8, CD14, CD19, IgG, etc.) as well as granulocyte subtype-specific transcripts were reasonably expressed by each leukocyte type as shown in FIGS. 6A-6R. Regarding the viability, we qualified the RNA before GeneChip assay using Array Quality Metrics Comparisons Software (Affymetrix) as well as trypan blue staining (they were always >95% viable), since RNAse-rich granules derived from degenerating cells rapidly destroy RNA transcripts. Briefly, to evaluate the quality of RNA, the ratio of 3′-probe set and 5′-probe set of housekeeping genes were compared as shown in FIGS. 6A-6R. According to the above software's guidance, the ratio of >2:1 at standard sample (5 μg total RNA) protocol and that of >10:1 at small sample (50 ng total RNA) protocol were recommended. As shown in FIGS. 6A-6R, the cells used in the present study had the appropriate ratios of 3′-probe set and 5′-probe set of housekeeping genes, suggesting that these cells were highly viable.

### Statistical Analysis

Since logarithmic “normalized AD” levels were normally distributed within each group, unpaired parametric Student's two-tailed t-test was employed to analyze the data on a logarithmic scale.

TABLE 1
Representative cell type-selective transcripts in granulocytes

| | | | |
|---|---|---|---|
| Accession #^{a} | Transcript | S.I.^{b} | Normalized AD level |

Basophil-selective

| | | | |
|---|---|---|---|
| NM_000589.1 | IL-4 | 73.3 | 13.3 |
| L35848.1 | HTm4 | 38.2 | 132.1 |
| BC005912.1 | Fc ε RI α | 12.7 | 218.9 |

Eosinophil-selective

| | | | |
|---|---|---|---|
| NM_001140.1 | Arachidonate | 74.1 | 18.3 |
| | 15-lipoxygenase | | |
| NM_024703.1 | FLJ22593 | 19.1 | 29.1 |
| NM_014442.1 | Siglec-8 | 9.8 | 16.9 |

Neutrophil-selective

| | | | |
|---|---|---|---|
| NM_004633.1 | IL-1 R, | 127.9 | 51.5 |
| | type II | | |
| U73191.1 | inward rectifier K⁺ | 107.5 | 98 |
| | channel Kir1.3 | | |
| NM_001557.1 | CXCR2 (IL-8 receptor β) | 39.3 | 105.2 |

Mast cell-selective

| | | | |
|---|---|---|---|
| AF206667.1 | tryptase β | 84.3 | 159.4 |
| NM_001911.1 | cathepsin G | 51.5 | 72.1 |
| BC005929.1 | major basic protein | 31.6 | 72.5 |

Basophil-eosinophil-selective

| | | | |
|---|---|---|---|
| M75914.1 | IL-5R α | 42.8 | 19.4(B), 29.3(E) |
| NM_004778.1 | CRTH2 | 16.6 | 23.9(B), 38.1(E) |
| NM_001828.3 | Charcot-Leyden | 15.2 | 229.2(B), 198.6(E) |
| | crystal protein | | |

Eosinophil-neutrophil-selective

| | | | |
|---|---|---|---|
| NM_005306.1 | GPR 43 (PAR1-like) | 21.7 | 11.7(E), 32.9(N) |
| NM_004668.1 | DHHC | 6.6 | 16.2(E), 44.5(N) |
| | domain containing 18 | | |

Basophil-neutrophil-selective

| | | | |
|---|---|---|---|
| NM_016006.1 | CGI-58 protein | 5.8 | 12.6(B), 21.2(N) |

Basophil-mast cell-selective

| | | | |
|---|---|---|---|
| NM_001870.1 | carboxypeptidase A3 | 59.2 | 111.7(M), 137.3(B) |
| NM_002529.2 | TRK neurotrophin | 34.7 | 3.1(M), 7 (B) |
| | receptor | | |
| NM_000139.1 | Fc ε RI β | 21.2 | 22.2(M), 43.8(B) |

^{a}The GenBank accession number (http://www.ncbi.nlm.nih.gov).
^{b}Selectivity index (S.I.) was calculated by comparing the “normalized AD” level in a cell type or of two cell types with the maximal gene expression level of the other 8 or 9 cell types. The complete list of the genes having >3 S.I. is shown in Supplementary Table 1.

FIGS. 6A-6R show the complete list of granulocyte subtype-selective transcripts. Selectivity index (S.I.) was calculated by comparing the “normalized AD” level of a cell type or of two cell types with the maximal gene expression level of the other 8 or 9 cell types including platelets (P1), CD4⁺ cells (CD4), CD8⁺ cells (CD8), CD14⁺ cells (CD14), CD19⁺ cells (CD19) and nasal polyp-derived cultured fibroblasts (Fb). Transcripts having S.I. >3-fold were shown in FIGS. 6A-6R A-H. A. Basophil (Ba)-selective transcripts. B. Eosinophil (Eo)-selective transcripts. C. Neutrophil (Ne)-selective transcripts. D. Mast cell (MC)-selective transcripts. E. Basophil and eosinophil-selective transcripts. F. Eosinophil and neutrophil-selective transcripts. G. Basophil and neutrophil-selective transcripts. H Mast cell and basophil-selective transcripts. I. Raw AD levels for the median values used to normalize the raw AD levels, and the housekeeping genes. When the result was accompanied by presence call, it was shown as a bold numeral. Italic numerals show that the raw AD levels were associated with absence call by the GeneChip analysis software. 1. Abbreviations used in the tables were (small); the results obtained by the small sample protocol (see materials and methods), R; receptor, and ICN; ion channel.

### REFERENCES

Useful techniques and compositions described in the references cited herein are incorporated herein by reference. In addition, U.S. Ser. No. 60/549,865, filed on Mar. 3, 2004, the benefit of the filing date of which is claimed under 35 U.S.C. §119(e), is incorporated herein by reference in its entirety.
1. Bochner B S. Systemic activation of basophils and eosinophils: markers and consequences. J Allergy Clin Immunol. 2000;106:S292-302.
2. Hirai K, Miyamasu M, Takaishi T, Morita Y. Regulation of the function of eosinophils and basophils. Crit Rev Immunol. 1997;17:325-52.
3. Savage C O. The evolving pathogenesis of systemic vasculitis. Clin Med. 2002;2:458-64.
4. Oehler R, Weingartmann G, Manhart N, et al. Polytrauma induces increased expression of pyruvate kinase in neutrophils. Blood. 2000;95:1086-92.
5. Church M K, Pao G J, Holgate S T. Characterization of histamine secretion from mechanically dispersed human lung mast cells: effects of anti-IgE, calcium ionophore A23187, compound 48/80, and basic polypeptides. J Immunol. 1982;129:2116-21.
6. Lin C S, Boltz R C, Blake J T, et al. Voltage-gated potassium channels regulate calcium-dependent pathways involved in human T lymphocyte activation. J Exp Med. 1993;177:637-45.
7. Saito H, Okajima F, Molski T F, Sha'afi R I, Ui M, Ishizaka T. Effects of ADP-ribosylation of GTP-binding protein by pertussis toxin on immunoglobulin E-dependent and -independent histamine release from mast cells and basophils. J Immunol. 1987;138:3927-34.
8. Ji T H, Grossmann M, Ji I. G protein-coupled receptors. I. Diversity of receptor-ligand interactions. J Biol Chem. 1998;273:17299-302.
9. Zambrowicz B P, Sands A T. Knockouts model the 100 best-selling drugs. Will they model the next 100 Nat Rev Drug Discov. 2003;2:38-51.
10. Venter J C, Adams M D, Myers E W et al. The sequence of the human genome. Science 2001;291:1304-51.
11. International Human Genome Sequencing Consortium. Initial sequencing and analysis of the human genome. Nature 2001;409: 860-921.
12. Drews J. Drug discovery: a historical perspective. Science. 2000;287:1960-4.
13. Hopkins A L, Groom C R. The druggable genome. Nature Rev Drug Discov. 2002;1:727-30.
14. Iida M, Matsumoto K, Tomita H, et al. Selective down-regulation of high-affinity IgE receptor (FcεRI) β-chain messenger RNA among transcriptome in cord blood-derived versus adult peripheral blood-derived cultured human mast cells. Blood. 2001;97:1016-22.
15. Nakajima T, Matsumoto K, Suto H, et al. Gene expression screening of human mast cells and eosinophils using high-density oligonucleotide probe arrays:

abundant expression of major basic protein in mast cells. Blood. 2001;98:1127-34.
16. Nakajima T, Inagaki N, Tanaka H, et al. Marked increase in CC chemokine gene expression in both human and mouse mast cell transcriptomes following Fcε receptor I cross-linking: an interspecies comparison. Blood. 2002;100:3861-8.
17. Yoshimura C, Miyamasu M, Nagase H, et al. Glucocorticoids induce basophil apoptosis. J Allergy Clin Immunol. 2001;108:215-20.
18. Matsumoto K, Schleimer R P, Saito H, Iikura Y, Bochner B S. Induction of apoptosis in human eosinophils by anti-Fas antibody treatment in vitro. Blood. 1995;86:1437-43.
19. Okayama Y, Hunt T C, Kassel O, Ashman L K, Church M K. Assessment of the anti-c-kit monoclonal antibody YB5.B8 in affinity magnetic enrichment of human lung mast cells. J Immunol Methods. 1994;169:153-61.
20. Fukagawa K, Okada N, Fujishima H, et al. CC-chemokine receptor 3: a possible target in treatment of allergy-related corneal ulcer. Invest Ophthalmol Vis Sci. 2002;43:58-62.
21. Arock M, Schneider E, Boissan M, Tricottet V, Dy M. Differentiation of human basophils: an overview of recent advances and pending questions. J Leukoc Biol. 2002;71:557-64.
22. Kepley C L, McFeeley P J, Oliver J M, Lipscomb M F. Immunohistochemical detection of human basophils in postmortem cases of fatal asthma. Am J Respir Crit Care Med. 2001;164:1053-8.
23. Loitto V-M, Forslund T, Sundqvist T, Magnusson K-E, Gustafsson M. Neutrophil leukocyte motility requires directed water influx. J Leukoc Biol. 2002;71:212-22.
24. Pfaffl M W. A new mathematical model for relative quantification in real-time RT-PCR. Nucleic Acids Res. 2001;29:e45.
25. Adra C N, Lelias J M, Kobayashi H, et al. Cloning of the cDNA for a hematopoietic cell-specific protein related to CD20 and the beta subunit of the high-affinity IgE receptor: evidence for a family of proteins with four membrane-spanning regions. Proc Natl Acad Sci USA. 1994;91:10178-82.
26. Adra C N, Mao X Q, Kawada H, et al. Chromosome 11q13 and atopic asthma. Clin Genet. 1999;55:431-7.
27. Donato J L, Ko J, Kutok J L, et al. Human HTm4 is a hematopoietic cell cycle regulator. J Clin Invest. 2002;109:51-8.
28. Nutku E, Aizawa H, Hudson S A, Bochner B S. Ligation of Siglec-8: a selective mechanism for induction of human eosinophil apoptosis. Blood. 2003;101:5014-20.
29. O'Reilly M, Alpert R, Jenkinson S, et al. Identification of a histamine H4 receptor on human eosinophils—role in eosinophil chemotaxis. J Recept Signal Transduct Res. 2002;22:431-48.
30. Hirai H, Tanaka K, Yoshie O, et al. Prostaglandin D2 selectively induces chemotaxis in T helper type 2 cells, eosinophils, and basophils via seven-transmembrane receptor CRTH2. J Exp Med. 2001;193:255-61.
31. Moffatt J D, Jeffrey K L, Cocks T M. Protease-activated receptor-2 activating peptide SLIGRL inhibits bacterial lipopolysaccharide-induced recruitment of polymorphonuclear leukocytes into the airways of mice. Am J Respir Cell Mol Biol. 2002;26:680-4.
32. Schmidlin F, Amadesi S, Vidil R, et al. Expression and function of proteinase-activated receptor 2 in human bronchial smooth muscle. Am J Respir Crit Care Med. 2001;164:1276-81.
33. Bradding P., Okayama Y., Kambe N., Saito H., Ion channel gene expression in human lung, skin and cord blood-derived mast cells, J. Leukoc Biol 2003; 73:614-20.
34. Gounni A S, Lamkhioued B., Koussih L., Ra C., Renzi P M, Hamid Q., Human neutrophils express the high-affinity receptor for immunoglobulin E (Fc epsilon RI): role in asthma. FASEB J. 2001; 15:940-9.
35. Koshino T., Teshima S., Fukushima N., Takaishi T., Hirai K., Miyamoto Y., et al. Identification of basophils by immunohistochemistry in the airways of post-mortem cases of fatal asthma. Clin Exp Allergy 1993; 23:919-25.
36. Kepley C. L., McFeeley P. J., Oliver J. M., Lipscomb M. F., Immunohistochemical detection of human basophils in postmortem cases of fatal asthma. Am J Respir Crit Care Med 2001; 164:1053-8.
37. Schroeder J. T., Lichtenstein L. M., Roche E. M., Xiao H., Liu M. C., Il-4 production by human basophils fund in the lung following segmental allergen challenge. J. Allergy Clin Immunol 2001; 107:265.71.
38. Luccioli S., Brody D. T., Hasan S., Keane-Myers A., Prussin C., Metcalfe D D, IgE(+), Kit (−), I-A/I-E-(−) myeloid cells are the initial source of Il-4 after antigen challenge in a mouse model of allergic pulmonary inflammation. J Allergy Clin Immunol 2001; 110:117-24.

We claim:

1. A method for diagnosing a granulocyte disorder comprising:
detecting in a biological sample from a subject a level of expression of one or more granulocyte-selective markers,
comparing the level of expression of each of the one or more granulocyte-selective markers with a reference level of expression, wherein a statistically significant difference between the level of expression of at least one granulocyte-selective marker and an expected level of expression for the at least one granulocyte-selective marker is indicative of a granulocyte disorder in the subject.

2. The method of claim 1, wherein the reference level of expression for a granulocyte-selective marker is a normal level of expression of the granulocyte-selective marker in a normal granulocyte.

3. The method of claim 1, wherein the biological sample is a blood sample.

4. The method of claim 1, wherein the biological sample is a tissue sample.

5. The method of claim 1, wherein the level of expression of each of the one or more granulocyte-selective markers is determined by determining an amount of an mRNA in the biological sample corresponding to each of the one or more granulocyte-selective markers.

6. The method of claim 5, wherein the method of determining the amount of mRNA comprises reverse transcription polymerase chain reaction (RT-PCR) amplification.

7. The method of claim 1, wherein the level of expression of each of the one or more granulocyte-selective markers is determined by determining an amount of a protein in the biological sample corresponding to each of the one or more granulocyte-selective markers.

8. The method of claim 7, wherein the method of determining the amount of a protein that corresponds to a granulocyte-selective marker comprises contacting the biological sample with an antibody that binds to the protein.

9. The method of claim 1, wherein a higher level of expression of at least one of the one or more granulocyte-selective markers in the biological sample compared to the expected level of expression for the at least one granulocyte-selective marker is indicative of the granulocyte disorder.

10. The method of claim 1, wherein a lower level of expression of at least one of the one or more granulocyte-selective markers in the biological sample compared to the expected level of expression for the at least one granulocyte-selective marker is indicative of the granulocyte disorder.

11. The method of claim 1, wherein the granulocyte disorder comprises an abnormally high number of one or more types of granulocyte in the biological sample.

12. The method of claim 1, wherein the granulocyte disorder comprises an abnormally low number of one or more types of granulocyte in the biological sample.

13. The method of claim 1, wherein the granulocyte disorder comprises an abnormal pattern of expression of one or more granulocyte selective markers in one or more types of granulocyte in the biological sample.

14. A method for diagnosing a non-neutrophil granulocyte disorder or mast cell disorder comprising:
detecting in a biological sample from a subject a level of expression of one or more non-neutrophil granulocyte or mast cell selective markers,
comparing the level of expression of each of the one or more non-neutrophil granulocyte or mast cell selective markers with a reference level of expression, wherein a statistically significant difference between the level of expression of at least one non-neutrophil granulocyte or mast cell selective marker and an expected level of expression for the at least one non-neutrophil granulocyte or mast cell selective marker is indicative of a non-neutrophil granulocyte disorder or mast cell disorder in the subject.

15. The method of claim 14, wherein the non-neutrophil granulocyte disorder is a basophil disorder.

16. The method of claim 15, wherein the basophil disorder is a basophil-associated tumor or cancer.

17. The method of claim 14, wherein the non-neutrophil granulocyte disorder is an eosinophil disorder.

18. The method of claim 17, wherein the eosinophil disorder is an eosinophil-associated tumor or cancer.

19. The method of claim 14, wherein the mast cell disorder is a mast cell-associated tumor or cancer.

20-48. (canceled)

49. An assay for identifying a compound that alters at least one physiological property of a granulocyte comprising:
contacting a granulocyte with a candidate compound that interacts with a granulocyte-selective marker,
determining at least one physiological property of the granulocyte after contact with the candidate compound,
comparing the at least one physiological property to one at least one reference property to determine whether the candidate compound alters at least one physiological property of the granulocyte.

50-71. (canceled)


SEQUENCE LISTING (31 sequences)

SEQ ID NO: 1 - DNA, 557 bp, Homo sapiens
tttttttttttgtggaaagatgataggtttatagtgactcaaaatattttagaaaaatttctgtagtgtcaagttctttcaaacttaaaattttaaccccagaggattttcgctgaataaatgagaattggctctatttcttctacttctggatagcccgagtaaaaatactaataatttctagattttagtggggaactacaattattaggacccatggatattgctgcagttcaaatacaatacagtaattacaaaatatagaccatctctttacaaatacaaattatagtatattacaagtcatgtacagtaaatctataattttaaacaaactagtgtatctaagtttacctggttgcgagtgcattattattccagtttacagttgcccttagcgtgacagtcagaaaccgaccatcggagtgatattctcttatgtaaactggcgtcacatcacagaaaaccttatttatgaggtcccattgccctcgcaataatcactggtagctgggttctgacttacttacacaccgtatttcagaacagctaaacag

SEQ ID NO: 2 - DNA, 1613 bp, Homo sapiens
  CDS (71)..(1198)
cgggttctacctgccttgaagaagacacctgacctgcggagtgagtgaccagtgtttccagagcctggcaatggatgccattcacatcggcatgtccagcacccccctggtgaagcacactgctggggctgggctcaaggccaacagaccccgcgtcatgtccaagagtgggcacagcaacgtgagaattgacaaagtggatggcatatacctactctacctgcaagacctgtggaccacagttatcgacatgaagtggagatacaaactcaccctgttcgctgccacttttgtgatgacctggttcctttttggagtcatctactatgccatcgcgtttattcatggggacttagaacccgatgagcccatttcaaatcataccccctgcatcatgaaagtggactctctcactggggcgtttctcttttccctggaatcccagacaaccattggctatggagtccgttccatcacagaggaatgtcctcatgccatcttcctgttggttgctcagttggtcatcacgaccttgattgagatcttcatcaccggaaccttcctggccaaaatcgccagacccaaaaagcgggctgagaccatcaagttcagccactgtgcagtcatcaccaagcagaatgggaagctgtgcttggtgattcaggtagccaatatgaggaagagcctcttgattcagtgccagctctctggcaagctcctgcagacccacgtcaccaaggagggggagcggattctcctcaaccaagccactgtcaaattccacgtggactcctcctctgagagccccttcctcattctgcccatgacattctaccatgtgctggatgagacgagccccctgagagacctcacaccccaaaacctaaaggagaaggagtttgagcttgtggtcctcctcaatgccactgtggaatccaccagcgctgtctgccagagccgaacatcttatatcccagaggaaatctactggggttttgagtttgtgcctgtggtatctctctccaaaaatggaaaatatgtggctgatttcagtcagtttgaacagattcggaaaagcccagattgcacattttactgtgcagattctgagaaacagcaactcgaggagaagtacaggcaggaggatcagagggaaagagaactgaggacacttttattacaacagagcaatgtctgatcacaggggcgccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgttagataaacagacatttagcaatgctgacattaaaaggaaatgtatttctatacaagattattagctgtaatacaagatatttatttaaccaatgaccttatggctgagagttgaattgtggttca

SEQ ID NO: 3 - protein, 375 aa, Homo sapiens [sequence omitted]

SEQ ID NO: 4 - DNA, 5397 bp, Homo sapiens
  CDS (384)..(1667)
gcgcactggagccctggccagcgcgcagccttcccggcgccggcgggctgggtcttgggaattctggtttgctttggctcactcgctttttacaaaccactggatcttacatgcctctgtaccccccacttccactccatgtccccatgctcctgcgccagcaacaggacatgttctctggatgtcagctgagtcattaaagtaactctgcatgtcagtagacagaccttggtagaaccacaaggctcccagagacacccatctctcctcatttttttggtgtgtgtgtcttcaccgaacattcaaaactgtttctccaaagcgttttgcaaaaactcagactgttttccaaagcagaagcactggagtccccagcagaagcgatgggcagtgtgcgaaccaaccgctacagcatcgtctcttcagaagaagacggtatgaagttggccaccatggcagttgcaaatggctttgggaacgggaagagtaaagtccacacccgacaacagtgcaggagccgctttgtgaagaaagatggccactgtaatgttcagttcatcaatgtgggtgagaaggggcaacggtacctcgcagacatcttcaccacgtgtgtggacattcgctggcggtggatgctggttatcttctgcctggctttcgtcctgtcatggctgttttttggctgtgtgttttggttgatagctctgctccatggggacctggatgcatccaaagagggcaaagcttgtgtgtccgaggtcaacagcttcacggctgccttcctcttctccattgagacccagacaaccataggctatggtttcagatgtgtcacggatgaatgcccaattgctgttttcatggtggtgttccagtcaatcgtgggctgcatcatcgatgctttcatcattggcgcagtcatggccaagatggcaaagccaaagaagagaaacgagactcttgtcttcagtcacaatgccgtgattgccatgagagacggcaagctgtgtttgatgtggcgagtgggcaatcttcggaaaagccacttggtggaagctcatgttcgagcacagctcctcaaatccagaattacttctgaaggggagtatatccctctggatcaaatagacatcaatgttgggtttgacagtggaatcgatcgtatatttctggtgtccccaatcactatagtccatgaaatagatgaagacagtcctttatatgatttgagtaaacaggacattgacaacgcagactttgaaatcgtggtcatactggaaggcatggtggaagccactgccatgacgacacagtgccgtagctcttatctagcaaatgaaatcctgtggggccaccgctatgagcctgtgctctttgaagagaagcactactacaaagtggactattccaggttccacaaaacttacgaagtccccaacactcccctttgtagtgccagagacttagcagaaaagaaatatatcctctcaaatgcaaattcattttgctatgaaaatgaagttgccctcacaagcaaagaggaagacgacagtgaaaatggagttccagaaagcactagtacggacacgccccctgacatagaccttcacaaccaggcaagtgtacctctagagcccaggcccttacggcgagagtcggagatatgactgactgattccttctctggaatagttactttacaacacggtctgttggtcagaggcccaaaacagttatacagatgacggtactggtcaagatgggtcaagcaagcggccacaagggactgaggcaagcacaatggtttcaaagaaagactgtaagctccatgattagcataaagcactaaccatgtctccatgtgacccgatggcacatagatgttgtagaataagttatgggtttttatgttttgttttgtgtttttccaaaacttgaacttgcaggcaagccttggttgggtatttgatttatccagaatgcttctctttagggaacaaggatgtttttaatggcataacaaaggcaagactctgccttaatttttgaaaagctgctaactacatgaacacaaatgtgtatttttgttgcagtgtagttttccttttgtgtaattttaaagtcagtgttgaattttattgaaagctcatgatgcgcttcaaagtggcaagtatttggctattaactgccaaaacaagagcctgattttttgaggccagtaattcgtttgctagaattgattttttttctctctctctttgttacataagggcattatgtaacactagccgaatggtagcctctgggttgttgtttttttcttttcctccatgatgttaatgggttatctcaaattttaagttaaactacctaaaataaataccaaagataatgcatatttttgcacagtggagcttacacttaaaagaaaacaaagccccatgggctgccttgaaatcaagagacaataactttgaacctcagcaagaccttgaaccgccggttcattttgcaccttattcagaaaatagagcatcatactcaccgagtctagtcagtgtagtgcttttaaaaattttgtcctttcatgtaacttttttattttaagaggaagaagaagaaaggggcacacacacacaataccgacgtctatcctttcctgctaggcagtgctggccaggctcatgtgtagtgtgcgagatggtgatgtactcttatatttttctgggcttttccttttgcacattccaaaattcatttcataagacaagatcttcataggacctccttggcatcctggcattctcaaaactgagccatccagcatgaaagataaatgggtttaaacccttgctgctgaatttattgcctggactgtcaggacatcaccagcccaccttcaccttagggaagatgccacacctggcctccacacttgctcttctgatcagtctgtctggattgagtcctacagtgtcagatagggcggcaaatgccaaagcagggaaacagggaggtgtggacaagccagtttgatgcagcacttcagatcaagtgcttaggaaggagaggaaacttgccttttttatggcagaggatagtaatgaaaatgtctcagtattttagggtcaatgagagccataaaaatataacataatcacaagtaaaggagataatggtctaaaacagctatttcccttttctgtgtgcatacttatgactgaatgtgagctaagcattttctcctgtggagccctagagcaggttactaaggaaggacacattgttttccagaagcctcccctgcctggctgactgccttgctagaaacataattttttttttctcactgaagctcaataatggaactctttttttttttttttttaatttaaagttccctatttgtgaattctgggattactgacttttctttttaattggagtctcaaaatcaactctcttatggtattatatctctgtatgccattaaaaaacagcttgttctagaatcatgtattttgtaaactgatgtttgtgatggtctctggttcttgaacagccatatctgaatgccgtgcctgcaaaactatgacaatttttgctgttttcagccttcagatttgatggcttgggaaactgaggtgttattttcaatgaaacaaagaaagagatgttaagcaagtggttgttttagatccaaatgtaaaggcaggtttgggaaggtgtttaaagagttggaggaattggggattgagttgtaaagaaaacttacagaagaggcaacaatttggttcttgacagtgagaggatattgagggcttcagctgctgctattatgatgttttgcaaaggaaaataatcaaaccaaagagtattcagtgatatgtaaattaaatgaagatacagtggagaatgggggtgaccacaaaagaggctccccctaaacacacagtgctgccacttaaaaagacttgagaaatttgaaagggggtgggtatggggggggcaagaaagagggagggaaatctttcaacttatttctgaaaaagagaaaaaaatataaaatttctggtgcacaggtttgttttttcaagaaaattttgcagaagctatgtttttaaagtgtacattttataaagtttatcagatattttcatatttaaagccaaatgtaaatagaggtctgtaaagaaaaataattgccatagaaagtataatttcagtgcagtaatttctgagagctagtacctatatgctaccggttagcatggttttagcaaatatataccagccttataaggttcgtattgctatgttcttctgttatttatttcagcatggactgttcatttgaaacctttttctagttattagcgttttaacagttacaagctttaaatggcaattttttttttttttttttttttttttttttttttgtcaagagccaagacacaggtaatgcacgacattgattgctgcattttaccttcaaaatatttgtccttattgactgggtctccttaattaatgtacacatgtcattagaatgcagacggaggggactcaccatgaatatctggggttgattcccagatgtgtgttgcttctctattgcaagcagattccctgttggatttacttcggatttattcccttttaaagaatttttgcccatatctggaagggcactatatttttgggaggagccatagattcctggttatcctatttttaaacaaaatgtagacaaagtgaactctattttgattattgagaaaggagtagttttctatccctctaagagtatacttgaatcagacattttaaggatgtcactatggcactgttgtcatttccaaattcctagaaaagtttgttttactttgtttttattctgttaatgcattctttcttctctttacttcctttcttaccagtacactcctatctcaactctgtttatttgatgagttctgtcccgtaaatcatatttcccttacaattaataaatgtcacttcatattttataataaaccactcagtaaaagcaaaagcttgtcctgagaagtagagtgagttctttttcactctgtgtctaataatgttaaggtgggaaaaaaaaaagtgtggcatagctacctgcccatccccaaccctcagcaaagtagaatctcttttctggtaattttgggtttccgctctgggctctggcaagttgaacaatcctagccattgacaatcgtgatagttattattttcccatttgctgtctttttgtatctaaagtcttcctattgtactgcacaaaccatggattgtacatatttttatatattatgtcttattttattatttctaaataaaaaaattaaaaattgaaaaaaaaaaaaa

SEQ ID NO: 5 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 6 - DNA, 2344 bp, Homo sapiens
  CDS (208)..(1380)
gaaggcgtggctccctcccgggccagtgagcctggcgccgccgcggccgcgtcccagcagcggagtagggcggcggctgcgccccgcaccatgggggcagcccagccccagccgcggtaaacgccgacctccgccgccgcccgcgcccgtctgccccctcccgctgcggctctctggacgccatcccctcctcacctcgaagccaacatgaaggagacccggggctacggaggggatgcccccttctgcacccgcctcaaccactcctacacaggcatgtgggcgcccgagcgttccgccgaggcgcggggcaacctcacgcgccctccagggtctggcgaggattgcggatcggtgtccgtggccttcccgatcaccatgctgctcactggtttcgtgggcaacgcactggccatgctgctcgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggccgtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctgataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccagatcaggtaccacacaaacaactatgcatccagctccacctccttaccctgccagtgttcctcaaccttgatgtggagcgaccatttggaaagataatgaaagaacggagttggacattttattgcaattcctgcttccctgaatttgcatatttcttcccacctgagaaggataattatatattttaatttggattatttcttcatttttatctttttattttaatgattgttttgtcagtaatacccatggagatcaactttattattataatccatgcctctgaatattagagggtttcttggatgggattttgaatatgcatttaagaacgttgggaacaatttcacagatgatgattggaggaaaagtgatgaaaagaaagacctgtgttccaggagttttctccaacttcaaacctttacgtgaatcttaaccaaagtggacatctttacatttcatgatagcttgcttttgcaatatgagtttgaaaaatcagtataagcttatgatggtgaaaagtcaacatattgagagtgataattcaattaataggatatgaacttaacgacatataaaagataaacttaacgacatataaaagcaaataagggcaggagggaatcgtgacaaaaaatatttgtgcccaattataaccaatgttttagagatgttgtgtccctgtggcttagcatggaactaaaagttctaagtctcaattctagttatgtgtcatttagtaactcaggaatctgcttaattgttatttctaagcttttgatgacaaaggagtgatgcagctaagggcatccttggagtgtcataaaaaacaaatttgaggttgaatgattagctgccagttagagtgataaaaaaatccaggtaggccttctgattcaccatgaccaagtcaggatttcttaatatttcttttctggcagcatatacaaaggcaaaattaataaataacagttgttgaataacaaactttattacgtttttataaaataaaagaatctattttgtctgtattaaaataaaaagctttgtggacttctaa

SEQ ID NO: 7 - protein, 390 aa, Homo sapiens
MKETRGYGGDAPFCTRLNHSYTGMWAPERSAEARGNLTRPPGSGEDCGSVSVAFPITMLLTGFVGNALAMLLVSRSYRRRESKRKKSFLLCIGWLALTDLVGQLLTTPVVIVVYLSKQRWEHIDPSGRLCTFFGLTMTVFGLSSLFIASAMAVERALAIRAPHWYASHMKTRATRAVLLGVWLAVLAFALLPVLGVGQYTVQWPGTWCFISTGRGGNGTSSSHNWGNLFFASAFAFLGLLALTVTFSCNLATIKALVSRCRAKATASQSSAQWGRITTETAIQLMGIMCVLSVCWSPLLIMMLKMIFNQTSVEHCKTHTEKQKECNFFLIAVRLASLNQILDPWVYLLLRKILLRKFCQIRYHTNNYASSSTSLPCQCSSTLMWSDHLER

SEQ ID NO: 8 - DNA, 3150 bp, Homo sapiens
  CDS (39)..(2699)
aaaagtttcttttctttgaatgacagaactacagcataatgcgtggcttcaacctgctcctcttctggggatgttgtgttatgcacagctgggaagggcacataagacccacacggaaaccaaacacaaagggtaataactgtagagacagtaccttgtgcccagcttatgccacctgcaccaatacagtggacagttactattgcgcttgcaaacaaggcttcctgtccagcaatgggcaaaatcacttcaaggatccaggagtgcgatgcaaagatattgatgaatgttctcaaagcccccagccctgtggtcctaactcatcctgcaaaaacctgtcagggaggtacaagtgcagctgtttagatggtttctcttctcccactggaaatgactgggtcccaggaaagccgggcaatttctcctgtactgatatcaatgagtgcctcaccagcagcgtctgccctgagcattctgactgtgtcaactccatgggaagctacagttgcagctgtcaagttggattcatctctagaaactccacctgtgaagacgtggatgaatgtgcagatccaagagcttgcccagagcatgcaacttgtaataacactgttggaaactactcttgtttctgcaacccaggatttgaatccagcagtggccacttgagtttccagggtctcaaagcatcgtgtgaagatattgatgaatgcactgaaatgtgccccatcaattcaacatgcaccaacactcctgggagctacttttgcacctgccaccctggctttgcaccaagcaatggacagttgaatttcacagaccaaggagtggaatgtagagatattgatgagtgccgccaagatccatcaacctgtggtcctaattctatctgcaccaatgccctgggctcctacagctgtggctgcattgcaggctttcatcccaatccagaaggctcccagaaagatggcaacttcagctgccaaagggttctcttcaaatgtaaggaagatgtgatacccgataataagcagatccagcaatgccaagagggaaccgcagtgaaacctgcatatgtctccttttgtgcacaaataaataacatcttcagcgttctggacaaagtgtgtgaaaataaaacgaccgtagtttctctgaagaatacaactgagagctttgtccctgtgcttaaacaaatatccacgtggactaaattcaccaaggaagagacgtcctccctggccacagtcttcctggagagtgtggaaagcatgacactggcatctttttggaaaccctcagcaaatatcactccggctgttcggacggaatacttagacattgagagcaaagttatcaacaaagaatgcagtgaagagaatgtgacgttggacttggtagccaagggggataagatgaagatcgggtgttccacaattgaggaatctgaatccacagagaccactggtgtggcttttgtctcctttgtgggcatggaatcggttttaaatgagcgcttcttcaaagaccaccaggctcccttgaccacctctgagatcaagctgaagatgaattctcgagtcgttgggggcataatgactggagagaagaaagacggcttctcagatccaatcatctacactctggagaacattcagccaaagcagaagtttgagaggcccatctgtgtttcctggagcactgatgtgaagggtggaagatggacatcctttggctgtgtgatcctggaagcttctgagacatataccatctgcagctgtaatcagatggcaaatcttgccgttatcatggcgtctggggagctcacgatggacttttccttgtacatcattagccatgtaggcattatcatctccttggtgtgcctcgtcttggccatcgccacctttctgctgtgtcgctccatccgaaatcacaacacctacctccacctgcacctctgcgtgtgtctcctcttggcgaagactctcttcctcgccggtatacacaagactgacaacaagatgggctgcgccatcatcgcgggcttcctgcactaccttttccttgcctgcttcttctggatgctggtggaggctgtgatactgttcttgatggtcagaaacctgaaggtggtgaattacttcagctctcgcaacatcaagatgctgcacatctgtgcctttggttatgggctgccgatgctggtggtggtgatctctgccagtgtgcagccacagggctatggaatgcataatcgctgctggctgaatacagagacagggttcatctggagtttcttggggccagtttgcacagttatagtgatcaactcccttctcctgacctggaccttgtggatcctgaggcagaggctttccagtgttaatgccgaagtctcaacgctaaaagacaccaggttactgaccttcaaggcctttgcccagctcttcatcctgggctgctcctgggtgctgggcatttttcagattggacctgtggcaggtgtcatggcttacctgttcaccatcatcaacagcctgcagggggccttcatcttcctcatccactgtctgctcaacggccaggtacgagaagaatacaagaggtggatcactgggaagacgaagcccagctcccagtcccagacctcaaggatcttgctgtcctccatgccatccgcttccaagacgggttaaagtcctttcttgctttcaaatatgctatggagccacagttgaggacagtagtttcctgcaggagcctaccctgaaatctcttctcagcttaacatggaaatgaggatcccaccagccccagaaccctctggggaagaatgttgggggcggtcttcctgtggttgtatgcactgatgagaaatcaggcgtttctgctccaaacgaccattttatcttcgtgctctgcaacttcttcaattccagagtttctgagaacagacccaaattcaatggcatgaccaagaacacctggctaccattttgttttctcctgcccttgttggtgcatggttctaagcatgcccctccagagcctatcatacgcctgatacagagaacctctcaataaatgatttgtcgcctgtctgactgatttaccctaggaaaaaaaaaaaaaaaaaaaaaaaaaaaa

SEQ ID NO: 9 - protein, 886 aa, Homo sapiens
MRGFNLLLFWGCCVMHSWEGHIRPTRKPNTKGNNCRDSTLCPAYATCTNTVDSYYCACKQGFLSSNGQNHFKDPGVRCKDIDECSQSPQPCGPNSSCKNLSGRYKCSCLDGFSSPTGNDWVPGKPGNFSCTDINECLTSSVCPEHSDCVNSMGSYSCSCQVGFISRNSTCEDVDECADPRACPEHATCNNTVGNYSCFCNPGFESSSGHLSFQGLKASCEDIDECTEMCPINSTCTNTPGSYFCTCHPGFAPSNGQLNFTDQGVECRDIDECRQDPSTCGPNSICTNALGSYSCGCIAGFHPNPEGSQKDGNFSCQRVLFKCKEDVIPDNKQIQQCQEGTAVKPAYVSFCAQINNIFSVLDKVCENKTTVVSLKNTTESFVPVLKQISTWTKFTKEETSSLATVFLESVESMTLASFWKPSANITPAVRTEYLDIESKVINKECSEENVTLDLVAKGDKMKIGCSTIEESESTETTGVAFVSFVGMESVLNERFFKDHQAPLTTSEIKLKMNSRVVGGIMTGEKKDGFSDPIIYTLENIQPKQKFERPICVSWSTDVKGGRWTSFGCVILEASETYTICSCNQMANLAVIMASGELTMDFSLYIISHVGIIISLVCLVLAIATFLLCRSIRNHNTYLHLHLCVCLLLAKTLFLAGIHKTDNKMGCAIIAGFLHYLFLACFFWMLVEAVILFLMVRNLKVVNYFSSRNIKMLHICAFGYGLPMLVVVISASVQPQGYGMHNRCWLNTETGFIWSFLGPVCTVIVINSLLLTWTLWILRQRLSSVNAEVSTLKDTRLLTFKAFAQLFILGCSWVLGIFQIGPVAGVMAYLFTIINSLQGAFIFLIHCLLNGQVREEYKRWITGKTKPSSQSQTSRILLSSMPSASKTG

SEQ ID NO: 10 - DNA, 2428 bp, Homo sapiens
  CDS (213)..(1229)
agtgttaccttggagcctacaatgagaggtatttcaaaatgagtgaagcatgactctcacagatgaaggcctagacgcaggatctttaatgaaaaaacacttgggccacttcaagacgacaaacgctcactgggcaaaacaccttcactgaaaagagacctcatattatgcaaaaaaaatcttaaaaggcctctgccttcagaagttacaagatgatcaattcaacctccacacagcctccagatgaatcctgctctcagaacctcctgatcactcagcagatcattcctgtgctgtactgtatggtcttcattgcaggaatcctactcaatggagtgtcaggatggatattcttttacgtgcccagctctgagagtttcatcatctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgacagatattataaaattgtaaagcctctttggacttctttcatccagtcagtgagttacagcaaacttctgtcagtgatagtatggatgctcatgctcctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcatcgtgtttgtgttttttgtctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccgtttagggaaatcttatgtaagaaattgcacattccattaaaagctcagaatgacctagacatttccagaatcaaaagaggaaatacaacacttgaaagcacagatactttgtgagttcctaccctcttccaaagaaagaccacgtgtgcatgttgtcatcttcaattacataacagaaatcaataagatatgtgccctcatcataaatatcatctctagcactgccatccaatttagttcaataaaattcaaatataagtttccatgcttttttgtaacatcaaagaaaacatacccatcagtaatttctctaatactgacctttctattctctattaataaaaaattaatacatacaattattcaattctattatattaaaataagttaaagtttataaccactagtctggtcagttaatgtagaaatttaaatagtaaataaaacacaacataatcaaagacaactcactcaggcatcttctttctctaaataccagaatctagtatgtaattgttttcaacactgtccttaaagactaacttgaaagcaggcacagtttgatgaagggctagagagctgtttgcaataaaaagtcaggtttttttcctgatttgaagaagcaggaaaagctgacacccagacaatcacttaagaaaccccttattgatgtatttcatggcactgcaaaggaagaggaatattaattgtatacttagcaagaaaattttttttttctgatagcactttgaggatattagatacatgctaaatatgttttctacaaagacttacgtcatttaatgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttactgtctatatactaataaagaaatgttttaataccgaaaaaaaaaaaaaaaa

SEQ ID NO: 11 - protein, 338 aa, Homo sapiens
MINSTSTQPPDESCSQNLLITQQIIPVLYCMVFIAGILLNGVSGWIFFYVPSSESFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 12 - DNA, 1734 bp, Homo sapiens
  CDS (85)..(1233)
ggcacgaggcgccgggccatgggcctcgagcccgccccgaacccccgcgagcccgccttgtctgcggcgtgactggaggcccagatggtcatcatgggccagtgctactacaacgagaccatcggcttcttctataacaacagtggcaaagagctcagctcccactggcggcccaaggatgtggtcgtggtggcactggggctgaccgtcagcgtgctggtgctgctgaccaatctgctggtcatagcagccatcgcctccaaccgccgcttccaccagcccatctactacctgctcggcaatctggccgcggctgacctcttcgcgggcgtggcctacctcttcctcatgttccacactggtccccgcacagcccgactttcacttgagggctggttcctgcggcagggcttgctggacacaagcctcactgcgtcggtggccacactgctggccatcgccgtggagcggcaccgcagtgtgatggccgtgcagctgcacagccgcctgccccgtggccgcgtggtcatgctcattgtgggcgtgtgggtggctgccctgggcctggggctgctgcctgcccactcctggcactgcctctgtgccctggaccgctgctcacgcatggcacccctgctcagccgctcctatttggccgtctgggctctgtcgagcctgcttgtcttcctgctcatggtggctgtgtacacccgcattttcttctacgtgcggcggcgagtgcagcgcatggcagagcatgtcagctgccacccccgctaccgagagaccacgctcagcctggtcaagactgttgtcatcatcctgggggcgttcgtggtctgctggacaccaggccaggtggtactgctcctggatggtttaggctgtgagtcctgcaatgtcctggctgtagaaaagtacttcctactgttggccgaggccaactcactggtcaatgctgctgtgtactcttgccgagatgctgagatgcgccgcaccttccgccgccttctctgctgcgcgtgcctccgccagtccacccgcgagtctgtccactatacatcctctgcccagggaggtgccagcactcgcatcatgcttcccgagaacggccacccactgatgactccaccctttagctaccttgaacttcagcggtacgcggcaagcaacaaatccacagcccctgatgacttgtgggtgctcctggctcaacccaaccaacaggactgactgactggcaggacaaggtctggcatggcacagcaccactgccaggcctccccaggcacaccactctgcccagggaatgggggctttgggtcatctcccactgcctgggggagtcagatggggtgcaggaatctggctcttcagccatctcaggtttagggggtttgtaacagacattattctgttttcactgcgtatccttggtaagccctgtggactggttcctgctgtgtgatgctgagggttttaaggtggggagagataagggctctctcgggccatgctacccggtatgactgggtaatgaggacagactgtggacaccccatctacctgagtctgattctttagcagcagagactgaggggtgcagagtgtgagctgggaaaggtttgtggctccttgcagcctccagggactggcctgtccccaatagaattgaagcagtccacggggaggggatgatacaaggagtaaacctttctttacactcaaaaaaaa

SEQ ID NO: 13 - protein, 382 aa, Homo sapiens
MVIMGQCYYNETIGFFYNNSGKELSSHWRPKDVVVVALGLTVSVLVLLTNLLVIAAIASNRRFHQPIYYLLGNLAAADLFAGVAYLFLMFHTGPRTARLSLEGWFLRQGLLDTSLTASVATLLAIAVERHRSVMAVQLHSRLPRGRVVMLIVGVWVAALGLGLLPAHSWHCLCALDRCSRMAPLLSRSYLAVWALSSLLVFLLMVAVYTRIFFYVRRRVQRMAEHVSCHPRYRETTLSLVKTVVIILGAFVVCWTPGQVVLLLDGLGCESCNVLAVEKYFLLLAEANSLVNAAVYSCRDAEMRRTFRRLLCCACLRQSTRESVHYTSSAQGGASTRIMLPENGHPLMTPPFSYLELQRYAASNKSTAPDDLWVLLAQPNQQD

SEQ ID NO: 14 - DNA, 993 bp, Homo sapiens
  CDS (1)..(993)
atgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccttgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtag

SEQ ID NO: 15 - protein, 330 aa, Homo sapiens
MLPDWKSSLILMAYIIIFLTGLPANLLALRAFVGRIRQPQPAPVHILLLSLTLADLLLLLLLPFKIIEAASNFRWYLPKVVCALTSFGFYSSIYCSTWLLAGISIERYLGVAFPVQYKLSRRPLYGVIAALVAWVMSFGHCTIVIIVQYLNTTEQVRSGNEITCYENFTDNQLDVVLPVRLELCLVLFFIPMAVTIFCYWRFVWIMLSQPLVGAQRRRRAVGLAVVTLLNFLVCFGPYNVSHLVGYHQRKSPWWRSIAVVFSSLNASLDPLLFYFSSSVVRRAFGRGLQVLRNQGSSLLGRRGKDTAEGTNEDRGVGQGEGMPSSDFTTE

SEQ ID NO: 16 - DNA, 1287 bp, Homo sapiens
  CDS (72)..(1085)
cctgtgtgccacgtgctggacaaatcttaactcctcaaggactcccaaaaccagagacaccaggagcctgaatggggaacgattctgtcagctacgagtatggggattacagcgacctctcggaccgccctgtggactgcctggatggcgcctgcctggccatcgacccgctgcgcgtggccccgctcccactgtatgccgccatcttcctggtgggggtgccgggcaatgccatggtggcctgggtggctgggaaggtggcccgccggagggtgggtgccacctggttgctccacctggccgtggcggatttgctgtgctgtttgtctctgcccatcctggcagtgcccattgcccgtggaggccactggccgtatggtgcagtgggctgtcgggcgctgccctccatcatcctgctgaccatgtatgccagcgtcctgctcctggcagctctcagtgccgacctctgcttcctggctctcgggcctgcctggtggtctacggttcagcgggcgtgcggggtgcaggtggcctgtggggcagcctggacactggccttgctgctcaccgtgccctccgccatctaccgccggctgcaccaggagcacttcccagcccggctgcagtgtgtggtggactacggcggctcctccagcaccgagaatgcggtgactgccatccggtttctttttggcttcctggggcccctggtggccgtggccagctgccacagtgccctcctgtgctgggcagcccgacgctgccggccgctgggcacagccattgtggtggggttttttgtctgctgggcaccctaccacctgctggggctggtgctcactgtggcggccccgaactccgcactcctggccagggccctgcgggctgaacccctcatcgtgggccttgccctcgctcacagctgcctcaatcccatgctcttcctgtattttgggagggctcaactccgccggtcactgccagctgcctgtcactgggccctgagggagtcccagggccaggacgaaagtgtggacagcaagaaatccaccagccatgacctggtctcggagatggaggtgtaggctggagagacattgtgggtgtgtatcttcttatctcatttcacaagactggcttcaggcatagctggatccaggagctcaatgatgtcttcattttattccttccttcattcaacagatatccatcatgcacttgctatgtgcaaggcctttttaggcactagagatatagcagtgaccaaaacagacacaaatcctgccc

SEQ ID NO: 17 - protein, 337 aa, Homo sapiens [sequence omitted]

SEQ ID NO: 18 - DNA, 2858 bp, Homo sapiens
  CDS (174)..(1175)
aacagtattttccttttcaacacatctattgaaagtgttggataaatgcaggatgttaatatgctataaacataaagtctgtttttaaaaaatagcatttgaaaatcatgaagggctttttgttttcttttgtttgtatatatgtttattggtaacaggtgacactggaagcaatgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatacgatcttgagcaacaaggaagcaacaccatcgtctgtgaaaaagtgtgcttccttaaaggggcctctggggctgaaatggcatcaaatggtaaataacatatgccagtttattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctgacaactgtacatagggttaacttctatttattgatgagacttccgtagataatgtggaaatcaaatttaaccaagaaaaaaagattggaacaaatgctctcttacattttattatcctcgtgtacagaaaagattatataaaatttaaatccacatagatctattcataagctgaatgaaccattactaagagaatgcaacaggatacaaatggccactagaggtcattatttctttctttctttttttttttttttaatttcaagagcatttcactttaacattttggaaaagactaaggagaaacgtatatccctacaaacctcccctccaaacaccttctcacattcttttccacaattcacataacactactgcttttgtgccccttaaatgtagatatgtgctgaaagaaaaaaaaaacgcccaactcttgaagtccattgctgaaaactgcagccaggggttgaaagggatgcagacttgaagagtctgaggaactgaagtgggtcagcaagacctctgaaatcctgggtaaaggattttctccttacaattacaaacagcctctttcacattacaataatataccataggaggcacaagcaccattattaagccactttgcttacaccttaagtgtgtacaattcaagtgtgagaatgctgtgttaactattctttggaattctccttctgtccagcaaatactctaatgatggttaaacatggcacctactcagcaatgccttcctggaccacaacccctatccccctgccccaccctcctcattaaaaacaaatacttctactgtttgggtgtgtgatagggttctcaatgcagatctcccttttctagttagctatattcttgactgcatccgctaaaaatgttaaagcttcttgagagacagacatgccagattttcttggtatctcccataatacgacctacagtccatggtctacagatgttttaaatagaattgctattctcgatacatacaaagacgtaattgctgacccacaatcagtaacatccatattgagagatttttcaaaggatggtgaccctgcttgtatttatttaccttggtattttttcttgcatccttctgtgattcaaaaaagtaaaatgtggctttctgaaatgatggataagagtctacatcttctagaaaaaatacataaaggagtagttaagctctgtaaatgtgccacgagctccaacacgaccatcgtagggtgaagcccacgttttcttccatggcctcaaaggccctagaacttgcctacctttctggccttacctcctagctacttatccatctcttgaactttatactcttgtataaatttctaactttcagaaaatgccatactctgttttggcaccacacatgtatatttccccctggtacacttggaagactcttatccatctgtgaaaccctatgttgtcatcacttggtccatgaaatattacctggccaatatcccaccatcacctcaaacccaatcaccccctcctctgtatgctgtcacacctatattattaaacttatcacattgcattgtaattacttcctgacctttgtatctactcttttagtaactgatgtatatatctgaaaggagagattgtttcattgtgcaatcaataaatgtttgataaaataaagccc

SEQ ID NO: 19 - protein, 333 aa, Homo sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWFFLFFISLPNTILSNKEATPSSVKKCASLKGPLGLKWHQMVNNICQFIFWTVFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG

SEQ ID NO: 20 - DNA, 878 bp, Homo sapiens
ttttttttttttttaagtggtaaaaaaaatttggttatttataaatcaattacacaataaaataattatttttaaaagtcacaaatacaatcgtggtataaagtcatttgggcatcaagtatctcttaaatatgtggcaaactatttgtccaaagagatgtggtccaaacccgtcgaaggctttataatttggtattagataacaaggtgaacaaaactgacaataaatactccaacgaattattttttaaaatactaaggggcaaaggctattctaaggggcaaaacaatctattactcagacctacctgaaaatttcacgtgaagtcgatcaaaagttatacaaaattggtatttacatgtttaaaatccggattggcatttttctttaataataatacatacaaaaactcagagggttaataaagaaataattcaaagtcctaataagtcaacaaacagatttcattataagctggaacataaaagagacaccatggttggctgtctcttttcaaaaattatcacggccacttggtcaaacgggaagcagcattcagaacaatggttctcaaatctcggggtggcataaacaccatgcaggttggttaccacacagattcctgggcctgttcccagtctgaagctctcataaaggatctgaggaatctgcttcgtgacaagattcagactatttatttatttatcacccaagctggagtgcaggggtgcaacccggtcaggaaacctcgcctcggagtaaaagggaaaccaaatcggggcccaggccctgagaatgggttcaaacgggcccaccaccggtaacggtatcagaaaaaaacggtccagaaggccaggggtcaaccgaaccaagaagc

SEQ ID NO: 21 - DNA, 1646 bp, Homo sapiens
  CDS (99)..(743)
tagtgatcttttctgagtgtctcctacttgcgacaaggtggacttgggaggaaagccgtctgccaaagcctgaagcctccaagccataaacaaccccaatggcctcccacgaagttgataatgcagagctggggtcagcctctgcccatggtaccccaggcagtgagacgggaccagaagagctgaatacttctgtctaccaccccataaatggatcaccagattatcagaaagcaaaattacaagttcttggggccatccagatcctgaatgcagcaatgattctggctttgggtgtctttctgggttccttgcaatacccataccacttccaaaagcacttctttttcttcaccttctacacaggctacccgatttggggtgctgtgtttttctgtagttcaggaaccttgtctgttgtagcagggataaaacccacaagaacatggatacagaacagttttggaatgaacattgccagtgctacaattgcactagtggggactgcttttctctcactaaatatagcagttaatatccagtcattaaggagttgtcactcttcatcagagtcaccggacctatgcaattacatgggctccatatcaaatggcatggtgtctctactgctgattctcaccttgctggaattatgcgtaactatctctaccatagccatgtggtgcaatgcaaactgctgtaattcaagagaggaaatttcctcacctcccaattctgtgtaatcaagaatacctccttatgaaaataattctgagagcatgaatatttgaccttaaatctccagtgactcagagcttcacccacaaactcaggagaacataagcctgctcgtaaagctcaatccttctatcatggcaccaatcacaagaaccttggacgtttgactgactctatcctttctctcctaactataaatcctatttgtgtgtcgtgggtatggaaggacagatatatttctttaggcattcttggatatctgtaacttctatgatcattactccaaagttgtttccagaaattggttctatttcttcttatccacctactccattgctttatgaggtttaaggaaggaaggcggtataatccctattcaatatattttttctaaaatccaacttctgaccgcccagtaggaagaaaaatgagacattttttccattacagagaaatgcttcttgactttaacatcagcattataaaaagtgtcaaataaaaaattaccatcattatcattaaaataaattttcactgtatttgagatgggagggttaaggctcagggattttatttcagtgaactgctggaactcacacatgccctgatatgtaaatgatgatttatgttggcgagtctgagagcaagcccaaatgtgttcttcaaaggacaatgggaaactgtaaagtagagaactaaagaataaggcctttagaatctgacacatctgggttcaaattctgaaactgtcacttattacctgtatgaacatgggcaaattatctaatctctctgatctatttttcctcatctgtaaaataggtgtaataataacaactactttgtcggttgctctgagggttaaatgaaaataaaaagaaaatgtgaaacagcaccacaggtacttg

SEQ ID NO: 22 - protein, 214 aa, Homo sapiens
MASHEVDNAELGSASAHGTPGSETGPEELNTSVYHPINGSPDYQKAKLQVLGAIQILNAAMILALGVFLGSLQYPYHFQKHFFFFTFYTGYPIWGAVFFCSSGTLSVVAGIKPTRTWIQNSFGMNIASATIALVGTAFLSLNIAVNIQSLRSCHSSSESPDLCNYMGSISNGMVSLLLILTLLELCVTISTIAMWCNANCCNSREEISSPPNSV

SEQ ID NO: 23 - DNA, 2481 bp, Homo sapiens
  CDS (179)..(1276)
ggccttgtcagctcacagcaggcgttaacagcctctaattgaggaaactgtggctggacaggttgcaaggcagttctgctccccatcgtcctcttgctgactggggactgctgagcccgtgcacggcagagagtctggtggggtggaggggctggcctggcccctctgtcctgtggaaatgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcctagttgctgcagcaattctcacctttcttgcacatcagcatctgctttgggaattggcacagtggatgacggcacaggagtctctatagaacagttcctagtctggagaggatatggaaatttgttcttgttctatattttgttttgaaaatgatgtctaacaaccatgataagagcaaggctgttaaataatatcttccaatttacagatcagacatgaatgggtggaggggttaggttgttcacaaaaggccacattccaagtatttgtaatctagaaagtgttatgtaagtgatgttattagcatcgagattccctccacctgattttcaagctgtcacttgtttccttttctcccctctctgggttgactgcatttctagactctcgccggcccaggcccatcttccaaagcaagaggaaggaatgataatggtgactcaggggaagaagaaacagccctcctctgaaagcctggactgtccggctgtgaactggctggcaggttctgcacgtgggtgggggccagggcctgggctttactcaattgcagagaaaaaactttctccctgcatctcatacctttacctctggccagttggccaccagggggagtgggctgaagggagagtagatggtgcaaagcaagcccatctctaagtagaaaaatcacccagagcacatgctgacctgataactggggtgttgagaccagctttgtccatggtatgatgtttgatttatgaagacgcattgttagaaatccatttggcttcttcatagaagtggcttcccagaggaagaggcctctcagaaaccatgttctatttaagttctgagtcctgatgagtgttccccaggatgcacattgaagggagggctcaggcagctgagggctgagaatgaggcagttggaatctagacactatgctgggttccctgagtcgtcaggccagacatttcaacaaggctgtggggagcagggctgtgactctggctgagcccaggaaagcgacaagggtgaactgggagaggacttactcagagaccccaacaggtgatactgcacaaagcctggttcttcaattttcctaccctgtatctaacataggagtttcatataaaacggtgatatcatgcagatgcagtctgaattccttgcctgaattaaatttatgtatcctctccaaaaaaaaaaaaaaaaaaa

SEQ ID NO: 24 - protein, 365 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 25 - DNA, 4574 bp, Homo sapiens
  CDS (593)..(3061)
gagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgaggcagcccgcgggcgtcatgcccgcgctcctccgcagcctggggtacgcgctgaagcccgggaggcttggcgccggcgaagacccaaggaccactcttctgcgtttggagttgctccccacaaccccgggctcgtcgctttctccatcccgacccagccggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaagcactcggggataaatagttccaatgcagaagtgctggctctgttcaatgtgaccgaggcggatgctggggaatatatatgtaaggtctccaattatatagggcaggccaaccagtctgcctggctcactgtcctgccaaaacagcaagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgattctcttcagtgaattttgataatggcttccagactctttgcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaactt

SEQ ID NO: 26 - protein, 822 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 27 - DNA, 5175 bp, Homo sapiens
  CDS (94)..(2676)
gccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgcccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggcctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggtgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaacaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtatggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatg

SEQ ID NO: 28 - protein, 860 aa, Homo sapiens [sequence omitted]

SEQ ID NO: 29 - DNA, 2070 bp, Homo sapiens
atgtttagggaggctctagggagaaatgcacagtttgacatcgttcatgaagagcctctccacggctcctgcgcctgagacagctggcctgacctccaaatcatccatccacccctgctgtcatctgttttcatagtgtgagatcaacccacaggaatatccatggcttttgtgctcattttggttctcagtttctacgagctggtgtcaggacagtggcaagtcactggaccgggcaagtttgtccaggccttggtgggggaggacgccgtgttctcctgctccctctttcctgagaccagtgcagaggctatggaagtgcggttcttcaggaatcagttccatgctgtggtccacctctacagagatggggaagactgggaatctaagcagatgccacagtatcgagggagaactgagtttgtgaaggactccattgcaggggggcgtgtctctctaaggctaaaaaacatcactccctcggacatcggcctgtatgggtgctggttcagttcccagatttacgatgaggaggccacctgggagctgcgggtggcagcactgggctcacttcctctcatttccatcgtgggatatgttgacggaggtatccagttactctgcctgtcctcaggctggttcccccagcccacagccaagtggaaaggtccacaaggacaggatttgtcttcagactccagagcaaatgcagatgggtacagcctgtatgatgtggagatctccattatagtccaggaaaatgctgggagcatattgtgttccatccaccttgctgagcagagtcatgaggtggaatccaaggtattgataggagagacgtttttccagccctcaccttggcgcctggcttctattttactcgggttactctgtggtgccctgtgtggtgttgtcatggggatgataattgttttcttcaaatccaaagggaaaatccaggcggaactggactggagaagaaagcacggacaggcagaattgagagacgcccggaaacacgcagtggaggtgactctggatccagagacggctcacccgaagctctgcgtttctgatctgaaaactgtaacccatagaaaagctccccaggaggtgcctcactctgagaagagatttacaaggaagagtgtggtggcttctcagggtttccaagcagggaaacattactgggaggtggacgtgggacaaaatgtagggtggtatgtgggagtgtgtcgggatgacgtagacagggggaagaacaatgtgactttgtctcccaacaatgggtattgggtcctcagactgacaacagaacatttgtatttcacattcaatccccattttatcagcctcccccccagcacccctcctacacgagtaggggtcttcctggactatgagggtgggaccatctccttcttcaatacaaatgaccagtcccttatttataccctgctgacatgtcagtttgaaggcttgttgagaccctatatccagcatgcgatgtatgacgaggaaaaggggactcccatattcatatgtccagtgtcctggggatgagacagagaagaccctgcttaaagggccccacaccacagacccagacacagccaagggagagtgctcccgacaggtggccccagcttcctctccggagcctgcgcacagagagtcacgccccccactctcctttagggagctgaggttcttctgccctgagccctgcagcagcggcagtcacagcttccagatgaggggggattggcctgaccctgtgggagtcagaagccatggctgccctgaagtggggacggaatagactcacattaggtttagtttgtgaaaactccatccagctaagcgatcttgaacaagtcacaacctcccaggctcctcatttgctagtcacggacagtgattcctgcctcacaggtgaagattaaagagacaacgaatgtgaatcatgcttgcaggtttgagggcacagtgtttgctaatgatgtgtttttatattatacattttcccaccataaactctgtttgcttattccaaaaaaaaaaaaaaaa

SEQ ID NO: 30 - DNA, 1696 bp, Homo sapiens
  CDS (88)..(1488)
ccgagtgtccacaccctgtgcgtctctctgtcctgccagcactgagggctcatccatccgcagagcagggcagtgggaggagacgccatgacccccatcctcacggtcctgatctgtctcgggctgagtctgggccccaggacccacgtgcaggcagggcacctccccaagcccaccctctgggctgagccaggctctgtgatcatccagggaagtcctgtgaccctcaggtgtcaggggagccttcaggctgaggagtaccatctatatagggaaaacaaatcagcatcctgggttagacggatacaagagcctgggaagaatggccagttccccatcccatccatcacctgggaacacgcagggcggtatcactgtcagtactacagccacaatcactcatcagagtacagtgaccccctggagctggtggtgacaggagcctacagcaaacccaccctctcagctctgcccagccctgtggtgaccttaggagggaacgtgaccctccagtgtgtctcacaggtggcatttgacggcttcattctgtgtaaggaaggagaagatgaacacccacaacgcctgaactcccattcccatgcccgtgggtggtcctgggccatcttctccgtgggccccgtgagcccgagtcgcaggtggtcgtacaggtgctatgcttatgactcgaactctccctatgtgtggtctctacccagtgatctcctggagctcctggtcccaggtgtttctaagaagccatcactctcagtgcagccaggtcctatggtggcccctggggagagcctgaccctccagtgtgtctctgatgtcggctacgacagatttgttctgtataaggagggagaacgtgacttcctccagcgccctggttggcagccccaggctgggctctcccaggccaacttcaccctgggccctgtgagcccctcccacgggggccagtacagatgctacagtgcacacaacctctcctccgagtggtcggcccccagtgaccccctggacatcctgatcacaggacagttctatgacagaccctctctctcggtgcagccggtccccacagtagccccaggaaagaacgtgaccctgctgtgtcagtcacgggggcagttccacactttccttctgaccaaggagggggcaggccatcccccactgcatctgagatcagagcaccaagctcagcagaaccaggctgaattccgcatgggtcctgtgacctcagcccacgtggggacctacagatgctacagctcactcagctccaacccctacctgctgtctctccccagtgaccccctggagctcgtggtctcagcatccctaggccaacacccccaggattacacagtggagaatctcatccgcatgggtgtggctggcttggtcctggtggtcctcgggattctgctatttgaggctcagcacagccagagaagcctacaagatgcagccgggaggtgaacagcagagaggacaatgcatacttcagcgtggtggagcctcagggacagatctgatgatcccaggaggctctggaggacaatctaggacctacattatctggactgtatgctggtcatttctagagacagcaatcaatatttgagtgtaaggaaactgtctggggtgattcctagaagatcattaaactgtggtacatttttttgtc

SEQ ID NO: 31 - protein, 466 aa, Homo sapiens
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSASLGQHPQDYTVENLIRMGVAGLVLVVLGILLFEAQHSQRSLQDAAGR